SEQUENCE LISTING (37 sequences)

SEQ ID NO: 1 - DNA, 116 bp, Artificial Sequence
  Description of Artificial Sequence Synthetic mutant of EGF1 (S53N) domain of human FVII
gatggggaccagtgtgcctcaaacccatgccagaatgggggctcctgcaaggaccagctccagtcctatatctgcttctgcctccctgccttcgagggccggaactgtgagacgca

SEQ ID NO: 2 - DNA, 116 bp, Artificial Sequence
  Description of Artificial Sequence Synthetic mutant of EGF1 (K62E) domain of human FVII
gatggggaccagtgtgcctcaagtccatgccagaatgggggctcctgcgaggaccagctccagtcctatatctgcttctgcctccctgccttcgagggccggaactgtgagacgca

SEQ ID NO: 3 - DNA, 116 bp, Artificial Sequence
  Description of Artificial Sequence Synthetic mutant of EGF1 (K62D) domain of human FVII
gatggggaccagtgtgcctcaagtccatgccagaatgggggctcctgcgacgaccagctccagtcctatatctgcttctgcctccctgccttcgagggccggaactgtgagacgca

SEQ ID NO: 4 - DNA, 116 bp, Artificial Sequence
  Description of Artificial Sequence Synthetic mutant of EGF1 (K62N) domain of human FVII
gatggggaccagtgtgcctcaagtccatgccagaatgggggctcctgcaacgaccagctccagtcctatatctgcttctgcctccctgccttcgagggccggaactgtgagacgca

SEQ ID NO: 5 - DNA, 116 bp, Artificial Sequence
  Description of Artificial Sequence Synthetic mutant of EGF1 (K62Q) domain of human FVII
gatggggaccagtgtgcctcaagtccatgccagaatgggggctcctgccaggaccagctccagtcctatatctgcttctgcctccctgccttcgagggccggaactgtgagacgca

SEQ ID NO: 6 - DNA, 116 bp, Artificial Sequence
  Description of Artificial Sequence Synthetic mutant of EGF1 (K62T) domain of human FVII
gatggggaccagtgtgcctcaagtccatgccagaatgggggctcctgcacggaccagctccagtcctatatctgcttctgcctccctgccttcgagggccggaactgtgagacgca

SEQ ID NO: 7 - DNA, 114 bp, Artificial Sequence
  Description of Artificial Sequence Synthetic mutant of EGF1 (P74A) domain of human FVII
gatggggaccagtgtgcctcaagtccatgccagaatgggggctcctgcaaggaccagctccagtcctatatctgcttctgcctcgctgccttcgagggccggaactgtgagacg

SEQ ID NO: 8 - DNA, 114 bp, Artificial Sequence
  Description of Artificial Sequence Synthetic mutant of EGF1 (A75D) domain of human FVII
gatggggaccagtgtgcctcaagtccatgccagaatgggggctcctgcaaggaccagctccagtcctatatctgcttctgcctccctgacttcgagggccggaactgtgagacg

SEQ ID NO: 9 - DNA, 114 bp, Artificial Sequence
  Description of Artificial Sequence Synthetic mutant of EGF1 (T83K) domain of human FVII
gatggggaccagtgtgcctcaagtccatgccagaatgggggctcctgcaaggaccagctccagtcctatatctgcttctgcctccctgccttcgagggccggaactgtgagaaa

SEQ ID NO: 10 - DNA, 116 bp, Artificial Sequence
  Description of Artificial Sequence Synthetic mutant of EGF1 (K62X) domain of human FVII
  modified_base (49)..(51): a, c, g, or t
gatggggaccagtgtgcctcaagtccatgccagaatgggggctcctgcnnngaccagctccagtcctatatctgcttctgcctccctgccttcgagggccggaactgtgagacgca

SEQ ID NO: 11 - protein, 38 aa, Artificial Sequence
  Description of Artificial Sequence Synthetic mutant of EGF1 (K62X) domain of human FVII
  MOD_RES (17): Any naturally occurring amino acid
DGDQCASSPCQNGGSCXDQLQSYICFCLPAFEGRNCET

SEQ ID NO: 12 - protein, 38 aa, Artificial Sequence
  Description of Artificial Sequence Synthetic mutant of EGF1 (S53N) domain of human FVII
DGDQCASNPCQNGGSCKDQLQSYICFCLPAFEGRNCET

SEQ ID NO: 13 - protein, 38 aa, Artificial Sequence
  Description of Artificial Sequence Synthetic mutant of EGF1 (K62E) domain of human FVII
DGDQCASSPCQNGGSCEDQLQSYICFCLPAFEGRNCET

SEQ ID NO: 14 - protein, 38 aa, Artificial Sequence
  Description of Artificial Sequence Synthetic mutant of EGF1 (K62D) domain of human FVII
DGDQCASSPCQNGGSCDDQLQSYICFCLPAFEGRNCET

SEQ ID NO: 15 - protein, 38 aa, Artificial Sequence
  Description of Artificial Sequence Synthetic mutant of EGF1 (K62N) domain of human FVII
DGDQCASSPCQNGGSCNDQLQSYICFCLPAFEGRNCET

SEQ ID NO: 16 - protein, 38 aa, Artificial Sequence
  Description of Artificial Sequence Synthetic mutant of EGF1 (K62Q) domain of human FVII
DGDQCASSPCQNGGSCQDQLQSYICFCLPAFEGRNCET

SEQ ID NO: 17 - protein, 38 aa, Artificial Sequence
  Description of Artificial Sequence Synthetic mutant of EGF1 (K62T) domain of human FVII
DGDQCASSPCQNGGSCTDQLQSYICFCLPAFEGRNCET

SEQ ID NO: 18 - protein, 38 aa, Artificial Sequence
  Description of Artificial Sequence Synthetic mutant of EGF1 (P74A) domain of human FVII
DGDQCASSPCQNGGSCKDQLQSYICFCLAAFEGRNCET

SEQ ID NO: 19 - protein, 38 aa, Artificial Sequence
  Description of Artificial Sequence Synthetic mutant of EGF1 (A75D) domain of human FVII
DGDQCASSPCQNGGSCKDQLQSYICFCLPDFEGRNCET

SEQ ID NO: 20 - protein, 38 aa, Artificial Sequence
  Description of Artificial Sequence Synthetic mutant of EGF1 (T83K) domain of human FVII
DGDQCASSPCQNGGSCKDQLQSYICFCLPAFEGRNCEK

SEQ ID NO: 21 - DNA, 26 bp, Artificial Sequence
  Description of Artificial Sequence Synthetic primer
gtgtgcctcaaacccatgccagaatg

SEQ ID NO: 22 - DNA, 22 bp, Artificial Sequence
  Description of Artificial Sequence Synthetic primer
gggctcctgcgaggaccagctc

SEQ ID NO: 23 - DNA, 22 bp, Artificial Sequence
  Description of Artificial Sequence Synthetic primer
gggctcctgcgacgaccagctc

SEQ ID NO: 24 - DNA, 22 bp, Artificial Sequence
  Description of Artificial Sequence Synthetic primer
gggctcctgcaacgaccagctc

SEQ ID NO: 25 - DNA, 22 bp, Artificial Sequence
  Description of Artificial Sequence Synthetic primer
gggctcctgccaggaccagctc

SEQ ID NO: 26 - DNA, 22 bp, Artificial Sequence
  Description of Artificial Sequence Synthetic primer
gggctcctgcacggaccagctc

SEQ ID NO: 27 - DNA, 23 bp, Artificial Sequence
  Description of Artificial Sequence Synthetic primer
gcttctgcctcgctgccttcgag

SEQ ID NO: 28 - DNA, 22 bp, Artificial Sequence
  Description of Artificial Sequence Synthetic primer
ctgcctccctgacttcgagggc

SEQ ID NO: 29 - DNA, 30 bp, Artificial Sequence
  Description of Artificial Sequence Synthetic primer
gccggaagtgtgagaaacacaaggatgacc

SEQ ID NO: 30 - DNA, 22 bp, Artificial Sequence
  Description of Artificial Sequence Synthetic primer
  modified_base (11)..(13): a, c, g, or t
gggctcctgcnnngaccagctc

SEQ ID NO: 31 - DNA, 28 bp, Artificial Sequence
  Description of Artificial Sequence Synthetic primer
cttacagtgatggtgaccagtgtgcctc

SEQ ID NO: 32 - DNA, 31 bp, Artificial Sequence
  Description of Artificial Sequence Synthetic primer
cggaactgtgagatgcataaggatgaccagc

SEQ ID NO: 33 - DNA, 34 bp, Artificial Sequence
  Description of Artificial Sequence Synthetic primer
ggtcgcaactgtgagaaacacaaggatgaccagc

SEQ ID NO: 34 - DNA, 27 bp, Artificial Sequence
  Description of Artificial Sequence Synthetic primer
tacaatgatggtgaccagtgtgcctcc

SEQ ID NO: 35 - DNA, 29 bp, Artificial Sequence
  Description of Artificial Sequence Synthetic primer
tcttatgcatctcacagttgcgaccctcg

SEQ ID NO: 36 - protein, 38 aa, Homo sapiens
DGDQCASSPCQNGGSCKDQLQSYICFCLPAFEGRNCET

SEQ ID NO: 37 - protein, 38 aa, Oryctolagus cuniculus
DGDQCASNPCQNGGSCEDQIQSYICFCLADFEGRNCEK

(19) United States
(12) Patent Application Publication
(10) Pub. No.: US 2007/0207960 A1
(43) Pub. Date: Sep. 6, 2007

(54) Mutants of the factor VII epidermal growth factor domain

(76) Inventors: Morris Blajchman, Hamilton (CA); Bryan Clarke, Ancaster (CA)

Correspondence Address: DAVID S. RESNICK, 100 SUMMER STREET, NIXON PEABODY LLP, BOSTON, MA, 02110-2131, US

(21) Appl. No.: 11/636,030

(22) Filed: Dec. 8, 2006

Related U.S. Application Data
(63) Continuation-in-part of application No. 11/294,670, filed on Dec. 5, 2005.
Application No. 11/294,670 is a continuation of application No. PCT/CA04/00826, filed on Jun. 3, 2004.
(60) Provisional application No. 60/475,905, filed on Jun. 5, 2003.

(51) Int. Cl. A61K 38/37

(52) U.S. Cl. 514/12; 530/383

(57) ABSTRACT

The application relates to modified blood coagulation factor, sequences encoding such modified factors, processes for their production, and related pharmaceutical compositions comprising such factors and their uses. More specifically, the application relates to mutations in the human FVII EGF-1 domain, wherein said mutations were analyzed for clotting activity, amidolytic activity and affinity of binding to full-length, relipidated human TF by competitive ELISA.

### TECHNICAL FIELD

The invention relates to mutants of Factor VII in the epidermal growth factor-like domain. The invention is further related to pharmaceutical compositions comprising such mutant factors and their uses.

### BACKGROUND OF THE INVENTION

Human factor VII (FVII) is a 406 amino acid [1] single-chain 50 kDa glycoprotein essential for the initiation of the blood coagulation cascade, as described in U.S. Pat. No. 5,580,560. FVII is synthesized in the liver and is secreted into the blood where it circulates predominately, approximately 98%, as an inactive zymogen precursor of activated factor VII (FVIIa), the serine protease that plays a key role in the initiation of the blood coagulation cascade. The binding of FVII to its specific cell-surface receptor tissue factor (TF), a calcium-dependent reaction of FVII with the transmembrane TF, converts the single-chain zymogen FVII to the two-chain enzymatically-active FVIIa form. The activation of FVII to FVIIa involves the hydrolysis of a single peptide bond between Arg152 and Ile153, thereby resulting in a two-chain molecule consisting of a light chain of 152 amino acids residues, and a heavy chain of 254 amino acid residues held together by a single disulfide bond.

FVII and FVIIa are multidomain proteins comprising an N-terminal Gla domain (γ-carboxyglutamic acid domain), which confers the ability of FVII or FVIIa to bind, in a reversible calcium-dependent manner to membranes containing negatively charged phospholipids, followed by two epidermal growth factor (EGF)-like domains, referred to as the EGF-1 and EGF-2, and a serine protease domain. These domains appear to be involved, to different extents, in an optimal interaction with TF.

Initiation of coagulation begins with the binding of either FVII or FVIIa to TF on the cell membrane. It is now well established that the first epidermal growth factor-like domain(EGF-1) of FVII is essential for the high affinity binding to TF [26, 15]. Analysis of the FVIIa-sTF, a complex of active site-inhibited human FVIIa with a protease-cleaved form of human soluble tissue factor (sTF), crystal structure [19] has shown that 70% of the binding energy between the two molecules involved amino acid residues in the EGF1 domain of FVII, yet this domain comprises only 38 of the 406 amino acids of human FVII. It should also be noted that the FVII-TF interaction (i.e. human FVII—human TF interaction) is one of high affinity, the K_{d} being variously estimated between 10⁻⁹ to 10⁻¹⁰ M.

The variable activity of TF from various animal tissues in the initiation of coagulation has been known [18]. Accordingly, it has been shown that the FVII-TF interaction is species-specific. For example, FVII from rabbits and FVII from mice both exhibited dramatically increased enzymatic activity with human TF when compared with homologous TF [18].

The FVII EGF-1 domain of FVII provides the region of greatest contact during the interaction of FVIIa with TF. Leonard et al. have shown that allosteric interaction(s) between the FVIIa active site (contained within the protease domain) and the EGF-1 domain is sensitive to variation in active site occupant structure, thereby indicating that the conformational change associated with FVII activation and active site occupation involves the EGF-1 domain [33]. Since the interaction of FVII with TF appears to play a critical role in coagulation, and other important biological processes, an understanding of the mechanisms by which the EGF-1 domain of FVII interacts with TF is of particular relevance and importance. Moreover, since the establishment of allosteric interaction(s) between the FVII EGF-1 and protease domains modulated both TF binding and the enzymatic activity of FVII, an examination of the FVII EGF-1 domain with a view to developing mutants of FVII with enhanced activity and/or affinity for TF would be of significant importance.

### SUMMARY OF THE INVENTION

The present invention fulfills a great need in the present art. The annual usage of recombinant FVIIa in Canada alone is estimated to be $20,000,000/annum. Mutants of human rFVIIa with increased affinity for TF and/or clotting activity could make the current use of wild-type rFVIIa obsolete. Furthermore, enzymatically-inactive forms of high-affinity rFVIIa mutants for TF could become novel anticoagulants.

An aim of the present invention is to provide FVII/FVIIa mutants with enhanced biological activity, enzymatic activity and/or binding affinity for TF. A preferred aim of the present invention is to provide human FVII/FVIIa mutants with enhanced biological activity, and more preferably, enhanced enzymatic activity and/or affinity for TF.

Accordingly, the present invention provides modified FVII/FVIIa mutants with enhanced biological activity, enzymatic activity and/or binding affinity for TF via site-directed mutagenesis of selected amino acids.

It has been determined that the increased clotting activity of rabbit FVII with human TF, compared to human FVII with human TF, may be explained by 5 non-conserved amino acid residues in the rabbit FVII EGF-1 versus the human FVII EGF-1 domain. More specifically, the 5 non-conserved amino acid residues are located at positions 53, 62, 74, 75, 83 of the EGF-1 domain, as illustrated in FIG. 1.

The present invention provides a modified factor VII/VIIa (also referred to herein as mutant FVII/FVIIa (or rFVII, rFVII, or rFVII/rFVIIa), preferably human FVII/FVIIa, comprising one or more mutation(s), wherein the mutation(s) is/are in the epidermal growth factor-like (EGF-1) domain.

More specifically, the present invention provides modified FVII/FVIIa comprising one or more mutation(s), wherein the mutation(s) is/are in the epidermal growth factor-like (EGF-1) domain, and in a preferred embodiment of the invention, the mutation(s) is/are at one, more than one, or all amino acid residues at residues 53, 62, 74, 75, 83, or any combination thereof, wherein the mutation may be to any amino acid residue that confers enhanced biological activity of FVII/FVIIa to, for example, positively improve blood coagulation, or increase affinity for TF. Accordingly, a mutation embodied by the present invention may be mutant FVII(K62x), wherein amino acid x is selected from any amino acid residue that increases the biological activity of FVII, such as the binding affinity of FVII for TF, or the clotting activity of FVII, or the amidolytic activity of FVII, or any functional activity that facilitates or improves the initiation of the blood coagulation cascade.

More particularly, the modified FVII/FVIIa mutants of the present invention comprise one, more than one or all mutations selected from (S53N), (K62E), (P74A), (A75D), or (T83K), or any combination thereof. In addition, the present invention also provides mutations K62D, K62N, K62Q, and K62T, wherein the presence of mutation K62T confers improved biological activity to the mutant rFVII(K62T) when compared to wild-type FVII. For example, a mutation embodied by the present invention may be mutant FVII(S53N)(K62E), FVII(K62T), FVII(S53N) (K62T), or FVII(K62E) (T83K), or any combination of mutations FVII(S53N), FVII(K62E), FVII(K62D), FVII(K62N), FVII(K62Q), and FVII(K62T), FVII(P74A), FVII(A75D), FVII(T83K) or any other mutation or combination of mutations at residues 53, 62, 74, 75, or 83 of in the EGF-1 of FVII. In a preferred embodiment of the invention, the modified FVII/FVIIa is FVII(K62E), where FVII(K62E) is a modified human FVII/FVIIa comprising a K to E mutation at amino acid residue 62. In another preferred embodiment of the invention, the modified FVII/FVIIa is FVII(K62T), where FVII(K62T) is a modified human FVII/FVIIa comprising a K to T mutation at amino acid residue 62. Table 2 below provides some FVII mutants with enhanced coagulant activity.

The present invention also provides a modified polypeptide, immunogenic polypeptide, or polypeptide fragment comprising a modified FVII/FVIIa according to the present invention, wherein said modified FVII/FVIIa more specifically comprises mutations of the EGF-1 domain, and preferably mutations at one, more than one, or all amino acid residues at positions 53, 62, 74, 75, 83, or any combination thereof.

A modified FVII/FVIIa according to the present invention provides enhanced biological activity, and more specifically enhanced enzymatic activity and/or affinity for TF.

The present invention also provides an isolated nucleotide comprising a sequence that encodes a purified polypeptide, immunogenic polypeptide, or polypeptide fragment of a modified FVII/FVIIa according to the present invention, wherein said modified FVII/FVIIa more specifically comprises mutation(s) within the EGF-1 domain, or mutations at one, more than one, or all amino acid residues at positions 53, 62, 74, 75, 83, or any combination thereof.

The invention also comprises recombinant nucleotide, or isolated nucleotide sequences encoding modified FVII/FVIIa according to the present invention, wherein said modified FVII/FVIIa more specifically comprises mutation(s) within the EGF-1 domain, or mutations at one, more than one, or all amino acid residues at positions 53, 62, 74, 75, 83, or any combination thereof, or any degenerate variant thereof.

The degeneracy of the genetic code is well known, wherein, most amino acid residues are encoded by more than one codon sequence, i.e. different codons can encode the same amino acid. Although certain nucleotide sequences noted herein encode specific codons to specific mutant amino acids in the various FVII mutants of the present invention, it is understood that other degenerate variant nucleotide sequences comprising differing codons for the equivalent mutant amino acids are also encompassed by the nucleotide sequences of the present invention. For example, mutant FVII(P74A) may be encoded by different but equivalent nucleotide sequences wherein mutant amino acid A, Alanine, may be encoded by different codons, such as GCA or GCC. Accordingly, primers directed towards the production of Ala may comprise different Ala codons, and will yield equivalent amino acid products. Accordingly, the nucleotide sequences of the present invention also comprise degenerate variants thereof.

In a preferred embodiment, the present invention comprises nucleotide sequence comprising a nucleotide sequence, for example, a cDNA or degenerate variant thereof, that encodes a modified FVII/FVIIa of the present invention, wherein said nucleotide sequence specifically hybridizes to a sequence selected from SEQ ID NO:1, SEQ ID NO:2, SEQ ID NO:3, SEQ ID NO:4, SEQ ID NO:5, or any degenerate variant thereof. SEQ ID NO:1 to SEQ ID NO: 4 are mutagenic primers of a preferred embodiment of the invention, wherein the highlighted codon encodes the mutagenic amino acid, and where said primer sequences bind to complementary nucleotide sequences, such as cDNAs, encoding modified FVII/FVIIa according to the present invention. Accordingly, the present invention also comprises mutagenic primers noted below, and any degenerate variants thereof, and additionally comprises nucleotide sequence and degenerate variants to the modified FVII/FVIIa that hybridize with said mutagenic primer.

S53→N

(SEQ ID NO:21)

(5′-GTGTGCCTCAAACCCATGCCAGAATG-3′)
K62→E

(SEQ ID NO:22)

(5′-GGGCTCCTGCGAGGACCAGCTC-3′)
K62→D

(SEQ ID NO:23)

(5′-GGGCTCCTGCGACGACCAGCTC-3′)
K62→N

(SEQ ID NO:24)

(5′-GGGCTCCTGCAACGACCAGCTC-3′)
K62→Q

(SEQ ID NO:25)

(5′-GGGCTCCTGCCAGGACCAGCTC-3′)
K62→T

(SEQ ID NO:26)

(5′-GGGCTCCTGCACGGACCAGCTC-3′)
P74→A

(SEQ ID NO:27)

(5′-GCTTCTGCCTCGCTGCCTTCGAG-3′)
A75→D

(SEQ ID NO:28)

(5′-CTGCCTCCCTGACTTCGAGGGC-3′)
T83→K

(SEQ ID NO:29)

(5′-GCCGGAAGTGTGAGAAACACAAGGATGACC-3′)
K62→X

(SEQ ID NO:30)

(5′-GGGCTCCTGCNNNGACCAGCTC-3′),

wherein NNN of SEQ ID NO:30 is a codon that encodes any amino acid that improves the biological activity of FVII, through the modification of residue 62 of the EGF-1 domain of FVII.

For example, in an embodiment of the invention, the mutations effected, at each codon are:

| | |
|---|---|
| SERINE53 (5′AGT-3′) TO ASPARAGINE | (5′AAC-3′) |
| LYSINE62 (5′AAG-3′) TO GLUTAMIC ACID | (5′GAG-3′) |
| LYSINE62 (5′AAG-3′) TO ASPARTIC ACID | (5′GAC-3′) |
| LYSINE62 (5′AAG-3′) TO ASPARAGINE | (5′AAC-3′) |
| LYSINE62 (5′AAG-3′) TO GLUTAMINE | (5′CAG-3′) |
| LYSINE62 (5′AAG-3′) TO THREONINE | (5′ACG-3′) |
| LYSINE62 (5′AAG-3′) TO THREONINE | (5′ACG-3′) |
| LYSINE62 (5′AAG-3′) TO ANY AMINO ACID | (5′NNN-3′) |
| PROLINE74 (5′CCT-3′) TO ALANINE | (5′GCT-3′) |
| ALANINE75 (5′GCC-3′) TO ASPARTIC ACID | (5′GAC-3′) |
| THREONINE83 (5′ACG-3′) TO LYSINE | (5′AAA-3′) |

As noted above, the present invention provides for any mutant FVII(K62x), or any FVII mutant comprising a mutation at K62, wherein amino acid x is selected from any amino acid residue that increases the biological activity of FVII, such as the binding affinity of FVII for TF, or the clotting activity of FVII, or the amidolytic activity of FVII, or any functional activity that facilitates or improves the initiation of the blood coagulation cascade. Although not all FVII K62 mutations are noted herein, the present invention embodies all K62 mutations, or any FVII mutants comprising a mutation at K62 of the EGF-1 domain, having improved or increased biological activity when compared to wild type FVII.

Accordingly, preferred embodiments of the present invention also comprise recombinant nucleotide sequences encoding modified FVII/FVIIa mutants according to the present invention, or any degenerate variant thereof. wherein said modified FVII/FVIIa more specifically comprises mutation(s) within the EGF-1 domain, or mutations at one, more than one, or all amino acid residues at positions 53, 62, 74, 75, 83, or any combination thereof.

Accordingly, the present invention also comprises corresponding nucleotide sequences encoding modified FVII/FVIIa mutants of the present invention and any degenerate variants thereof. The Sequence listings provided for the nucleotide and amino acid sequences of the FVII/FVIIa mutants comprises the sequence of the EGF-1 domain, however it is understood that the present invention embodies the functional full length FVII/FVIIa mutant, or any functional fragment thereof, where the modified EGF-1 domain of said modified FVII/FVIIa mutant is provided herein. Accordingly, the present invention provides: SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, wherein SEQ ID NO:1-10 refer to nucleotide sequences, wherein degenerate equivalents are also embodied herein, of the EGF-1 domain of the mutant FVII(xABy). Accordingly the present invention embodies any FVII mutant sequence comprising the nucleotide sequence of the EGF-1 domain comprising a sequence of any one of SEQ ID NO:1-10 or any degenerate equivalent thereof. The present invention also comprises any vector comprising the FVII mutant sequences embodied herein, wherein said vector may be an expression vector, preferably pCMV5, or a cloning vector, preferably pUC19. Also provided is a culture cell, cell or cell line, preferably HEK293, CHO or BHK cells or any related cell or progeny thereof, wherein said culture cell, cell or cell line is transfected with a FVII modified nucleotide sequences, wherein said modified nucleotide sequences comprises a modified EGF-1 domain sequence of any one of SEQ ID NO:1-10.

The present invention also provides SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, and SEQ ID NO:11, which correspond to the amino acid sequence of the modified EGF-1 domain comprised in the modified FVII/FVIIa(xABy) protein, or functional fragment thereof embodied herein, as provided by the nucleotide sequence of SEQ ID NO: 1-10. Accordingly the present invention embodies any FVII mutant sequence comprising the amino acid sequence of the EGF-1 domain comprising a sequence of any one of SEQ ID NO:11-20 or any functional variant or equivalent thereof. It should be further noted that the present invention embodies the functional full length FVII/FVIIa mutant, or any functional fragment or equivalent thereof, where the modified EGF-1 domain of said modified FVII/FVIIa mutant is provided herein, as specified in SEQ ID NO:11-20. The modified FVII/FVIIa(xABy) of the present invention may be produced as full length or as biologically active functional fragments thereof, wherein the mutant protein or polypeptide embodied herein exhibits improved biological activity compared to the FVII/FVIIa wild type. The modified FVII mutants embodied herein may be expressed, isolated and purified according to known protein and polypeptide procedures.

Also provided are the mutagenic primers having SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, and SEQ ID NO:30, wherein SEQ ID NO:21-28 comprise mutagenic primers that hybridize to the nucleotide sequences of SEQ ID NO:1-10, respectively.

In a preferred embodiment, the present application provides modified FVII/FVIIa, or functional fragment thereof, comprising one or more mutation(s), wherein said mutation(s) are in the EGF-1 domain, and are preferably any one, more than one, or all residues 53, 62, 74, 75, or 83 of the EGF-1 of FVII/FVIIa, provided that said mutation at residues 53, 62, 74, 75, or 83 results in a modified FVII/FVIIa having improved biological activity with respect to the wild type FVII. More preferably, the modified FVII mutant comprises a mutation at K62 of the EGF-1 domain, wherein the mutation at K62 confers enhanced biological activity.

In an embodiment of the present invention there is provided a modified FVII/FVIIa protein, or biologically active fragment thereof, wherein the modified FVII/FVIIa comprises mutation(s) at any one of residues 53, 62, 74, 75, or 83 of the EGF-1, or any combination thereof. In a preferred embodiment, the present invention provides a modified FVII/FVIIa(K62x) protein, or biologically active fragment thereof, and more preferably a modified FVII/FVIIa(K62E) protein or functional equivalent thereof.

It is additionally noted that the present invention also comprises any degenerate variants of the nucleotide sequences according to the present invention. Moreover, the present invention also comprises cDNA nucleotide sequences and degenerate variants, that encode modified FVII/FVIIa, wherein said modified FVII/FVIIa more specifically comprises mutation(s) within the EGF-1 domain, or mutations at one, more than one, or all amino acid residues at positions 53, 62, 74, 75, 83, or any combination thereof.

According to a preferred embodiment, the present invention comprises a nucleotide sequence comprising any one of SEQ ID NO:1-10, or any nucleotide sequence that comprises a sequence(s) encoding one, more than one, or all mutations at residues 53, 62, 74, 75, 83 of the EGF1 domain of FVII/FVIIa, or any combination thereof, or any degenerate variant thereof, that yields a modified FVII/FVIIa according to the present invention. Therefore, the present invention additionally comprises a nucleotide sequence, such as a cDNA, that encodes for a modified FVII/FVIIa mutant comprising mutation(s) at one, more than one, or all amino acid residues at positions 53, 62, 74, 75, 83, of human FVII/FVIIa, or any combination thereof.

The present invention comprises nucleotide sequence that encodes a polypeptide with the amino acid sequence of FIG. 1, or a polypeptide comprising any single or individual mutation contained therein, or any multiple mutations, or any combinations thereof.

The present invention also provides a vector comprising a nucleotide sequence encoding a modified FVII/FVIIa according to the present invention, and more specifically, wherein said modified FVII/FVIIa comprises mutation(s) within the EGF-1 domain, or mutations at one, more than one, or all amino acid residues at positions 53, 62, 74, 75, 83, or any combination thereof.

The present invention also provides vectors comprising all the various nucleotide sequences of the present invention, wherein said nucleotide sequences encode a modified FVII/FVIIa according to the present invention, or equivalents thereof, such as protein fragments, or (poly)peptides, or other equivalent functional molecules. In a preferred embodiment, a vector of the present invention may be an expression vector, preferably pCMV5, or a cloning vector, preferably pUC19.

In accordance with the present invention there is provided a culture cell or cell line transfected with a vector according to the present invention, or a progeny of said cell, wherein said culture cell or cell line, preferably HEK293, CHO, or BHK cells, or any other cell of human or non human origin suitable for the expression of a modified FVII/FVIIa of the present invention, or for the pharmaceutical, clinical or therapeutic uses thereof, wherein said cell or cell line is capable of expressing modified FVII/FVIIa according to the present invention or functional equivalents thereof.

The present invention also provides a culture cell or cell line that permanently expresses a mutant or modified nucleotide sequence of a modified FVII/FVIIa according to the present invention, wherein said culture cell or cell line is transfected with a vector according to the present invention and, may be preferably co-transfected with a selection plasmid, such as pSV2neo.

In a preferred embodiment, the present invention provides a culture cell or cell line that expresses a modified FVII/FVIIa according to the present invention, wherein the cell is preferably HEK293, CHO, or BHK cells, or any other cell of human or non human origin suitable for the expression of a modified FVII/FVIIa of the present invention, or for the pharmaceutical, clinical or therapeutic uses thereof, wherein said cell or cell line is capable of expressing modified FVII/FVIIa according to the present invention or functional equivalents thereof. In a preferred embodiment of the present invention, an HEK293, CHO or BHK culture cell or cell line that expresses a modified FVII(K62E) or FVII(A75D), or any other modified FVII/FVIIa according to the present invention, that is, any FVII/FVIIa (AxyB) molecule, and preferably any human FVII/FVIIa (AxyB) molecule, wherein A is a wild type amino acid, xy is the location of said amino acid, preferably comprised in the EGF-1 domain, and more preferably in the EGF-1 domain of human FVII/FVIIa, and more specifically at one, more than one, or all amino acid residues at positions 53, 62, 74, 75, 83, or any combination thereof, where B is said mutant amino acid.

The present invention also provides a cell, or cell line comprising the recombinant nucleotide molecule encoding a mutant FVII according to the present invention. According to a preferred embodiment, the present invention provides a cultured cell, or cell line comprising a vector according to the present invention, wherein said culture cell or cell line is preferably HEK293, CHO or BHK CELLS.

Moreover, the present invention also comprises HEK293 cell lines which permanently express recombinant FVII/FVIIa mutants. In a preferred embodiment, the mutant nucleotide sequences, or cDNAs, of mutants FVII(K62E), and/or FVII(A75D), or other modified FVII/FVIIa of the present invention, were subcloned into an expression vector, preferably pCMV5 expression vector and co-transfected into a cell, preferably HEK293 cell line along with a selection plasmid, preferably pSV2neo. Although permanent cell lines expressing mutants K62E and A75D have been established, the present invention also comprises the establishment of other permanent cell lines including CHO and BHK cells to other FVII/FVIIa mutants according to the present invention.

According to a preferred embodiment of the present invention, there is provided human FVII mutants wherein mutations considered are, in the EGF-1 domain, and more specifically at positions 53, 62, 74, 75, 83, or any combination thereof, wherein residues S53, K62, P74, A75, T83 may be mutated to any amino acid residue that increases the biological activity of FVII/FVIIa and preferably modified human FVII/FVIIa, to positively improve blood coagulation.

In a preferred embodiment of the present invention, there is provided a modified or mutant protein factor, or equivalent thereof, wherein said protein factor is human FVII/FVIIa, and where at least one amino acid in the EGF-1 domain. In a preferred embodiment, a modified or mutant protein factor, or equivalent thereof comprises mutation(s) preferably at residue positions 53, 62, 74, 75, 83, has been substituted with another amino acid which confers increased functional activity, such as binding affinity to TF, or clotting affinity, to said mutant factor. In a preferred embodiment, one, more or all the amino acid residues at S53, K62, P74, A75, T83 have been mutated to FVII(S53N), FVII(K62E), FVII(K62D), FVII(K62N), FVII(K62Q), FVII(K62T), FVII(P74A), FVII(A75D), FVII(T83K), or any combination thereof.

Accordingly, it should be further noted that the present invention is contemplated to cover any combination of mutations at any amino acid residue comprised in the EGF-1 domain, and more preferably at residues S53, K62, P74, A75, T83 of human FVII/FVIIa.

The invention is further directed to a method of expressing a modified factor VII/VIIa according to the present invention or equivalent thereof, in a cell, preferably cell line HEK293, wherein the method comprises: providing an expression vector, preferably pCMV5, encoding the modified protein; introducing the vector into the cell; and maintaining the cell under conditions permitting the expression of the protein in the cell. The method of the present invention also provides for the expression of the modified FVII/FVIIa in vivo or in vitro in other permanent cell lines.

In a preferred embodiment of the present invention, the present invention provides mutant human FVII(K62E), wherein the mutant factor exhibits significantly increased clotting activity when compared to plasma-derived human FVII/FVIIa. Combinations of other FVII mutants according to the invention, such as FVII(K62E), and FVII(A75D) aim to further increase the biological activity of the FVII, and preferably of human FVII.

The present invention also comprises a cell transformed with a recombinant nucleotide molecule comprising an isolated nucleotide sequence, and degenerate variants thereof, encoding a mutant or modified FVII protein or equivalent thereof, wherein said mutations are at one or more than one residue of the EGF-1 domain, and more preferably at one, more than one, or all residues 53, 62, 74, 75, or 83 or combinations thereof. Mutant FVIIa can be obtained by activating mutant FVII.

There is also provided an expression vector and a cloning vector encoding modified human FVII cDNA, wherein said expression vector is preferably pCMV5 or another suitable expression vector, said cloning vector is preferably pUC19 or another suitable expression vector, and wherein said cDNA is nucleotide sequence encoding a modified FVII/FVIIa, and preferably a modified human FVII/FVIIa, wherein the mutation to FVII may be any mutation according to the present invention. More specifically, said mutation may be at any amino acid comprising the EGF-1 domain of FVII, and preferably at any one of, more than one, or all amino acid residues 53, 62, 74, 75 and 83 or any combination thereof.

The present invention also provides a pharmaceutical composition comprising a modified FVII/FVIIa mutant product, or equivalent thereof, such as a functional peptide fragment, or other fragment, according to the present invention, or complexes of said modified FVII/FVIIa and a pharmaceutically accepted carrier.

The present invention further provides a method of treating a patient with condition or disorder, such as a bleeding disorder, or treating patients with thrombocytopenia, wherein the method comprises introducing into the patient a pharmaceutically effective amount of a modified FVII/FVIIa, and preferably a modified human FVII/FVIIa according to the invention, or any functional equivalent thereof, or an expression vector encoding a modified human FVII protein, such that an amount of the modified protein is effective to improve blood coagulation. In another embodiment of the method of the present invention, there is provided a modified human FVII with increased binding affinity for TF, or a modified human FVII-TF complex, wherein the amounts of the modified FVII protein, or the complexed modified FVII are in amounts effective to improve blood coagulation.

The present invention provides a method of treating a patient, preferably a patient with a bleeding condition or other blood related condition, such as patients with thrombocytopenia or other conditions, where said method comprises administration of a pharmaceutically effective amount of a modified FVII/FVIIa according to the invention, wherein said modified FVII/FVIIa more specifically comprises mutation(s) within the EGF-1 domain, or mutations at one, more than one, or all amino acid residues at positions 53, 62, 74, 75, 83, or any combination thereof.

The present invention also provides pharmaceutical compositions comprising various combinations of modified FVII/FVIIa according a preferred embodiment of the present invention, wherein one or more various differing FVII/FVIIa mutants may comprise a single pharmaceutical composition, wherein such mutants may comprise mutations that are preferably comprising the EGF-1 domain, or at one, more than one, or all amino acid residues at positions 53, 62, 74, 75, 83, or any combination thereof. Such pharmaceuticals are in accordance with the embodiments of the present invention and provide for increased synergistic biological effectiveness.

There is also provided a pharmaceutical composition comprising modified FVII/FVIIa, or any products thereof, such as nucleotide or amino acid products, such as mutant proteins or peptides according to the present invention, or sequences comprising the corresponding mutant FVII sequence or equivalents thereof, according to the present invention, or complexes of said modified FVII/FVIIa and a pharmaceutically accepted carrier and pharmaceutically acceptable vehicles, such as lipid encapsulation vesicles.

A pharmaceutical composition of the present invention comprises modified FVII/FVIIa according to the present invention, or complexes of modified FVII/FVIIa and a pharmaceutically accepted carrier. Preferably, a pharmaceutical composition according to the present invention may comprise a mutant FVII factor, such a FVII(K62E), FVII(K62T), or any mutant combination of FVII, such as mutation(s) comprising the EGF-1 domain with mutation(s) at residues one or more or any combination of mutations at residues 53, 62, 74, 75, 83, or any vector encoding the same, or any cell comprising the sequence information and cellular machinery and conditions permitting the expression of said mutant factors.

The present invention also provides a method for treating a patient with a bleeding disorder, or any blood related condition, such as patients with thrombocytopenia, comprising introducing into the patient, the FVII mutant peptide or protein, a vector, preferably an expression vector encoding a modified FVII/FVIIa according to a preferred embodiment of the present invention, in a pharmaceutically effective amount.

In a preferred embodiment, a preferred pharmaceutical composition of the present invention comprises an effective amount of mutant FVII mutant protein, or peptide, wherein said FVII mutant is as embodied in the present invention. In accordance with a preferred treatment of the present invention, a patient is provided with a pharmaceutically effective amount of a pharmaceutical composition according to the present invention.

A method for treating a patient with a pharmaceutical composition according to the present invention, wherein said modified FVII/FVIIa may be complexed to another molecule, or may be encapsulated in an acceptable vehicle, such as a lipid vesicle.

In another embodiment, the present invention additionally provides a strategy for selecting amino acid residues for mutagenesis, wherein said method aims to produce mutants with enhanced biological activity, or modulated enzymatic activity, wherein the method comprises: comparison of enzymatic activity of related interspecies native enzymes or protein for a specific substrate or antigen; comparison of the nucleotide or amino acid sequences of said native enzymes or proteins with enhanced or altered activities; determination of the non-conserved nucleotide or amino acid sequences between said native enzymes or proteins; specific modification of said non-conserved nucleotide or amino acid residues to yield mutant enzymes or proteins; determination of change in biological activity of said enzymes with respect to said native enzymes or proteins; and the expression and purification of said mutant enzymes or proteins.

Accordingly, there is provided a method for making mutants with enhanced or modulated enzymatic activity, wherein said method comprises: a) comparison of enzymatic activity of related interspecies native enzymes or protein for a specific substrate or antigen; b) comparison of the nucleotide or amino acid sequences of said native enzymes or proteins with enhanced or altered activities; c) determination of the non-conserved nucleotide or amino acid sequences between said native enzymes or proteins; d) specific modification of said non-conserved nucleotide or amino acid residues to yield mutant enzymes or proteins; e) determination of change in biological activity of said enzymes with respect to said native enzymes or proteins; f) expression and purification of said mutant enzymes or proteins. In a preferred embodiment, said modification is via site-directed mutagenesis. In another preferred embodiment, said modification may be at single points, or any more than one loci. Furthermore, said modification(s) may yield a mutant library, where said library comprises mutant enzymes or proteins with mutations at any one of said non-conserved nucleotide or amino acid residue for the mutant libraries may be generated.

In a preferred embodiment of the above noted mutation strategy method, modification may be effected via site-directed mutagenesis, at single amino acid residues, or more than one residue loci.

In another embodiment of the above noted mutant strategy method of the present invention, there is provided a method wherein modification(s) may yield a mutant library, where said library comprises mutant enzymes or proteins with mutations at any one of said non-conserved nucleotide or amino acid residue for the mutant libraries may be generated.

In a preferred embodiment of the present invention, there is provided a FVII/FVIIa mutant library, and more preferably a human FVII/FVIIa mutant library comprising various FVII/FVIIa mutants, wherein mutations to FVII/FVIIa are at one or more residue(s) comprising the EGF-1 domain. In a more preferred embodiment, the mutant library of the present invention may comprise FVII mutants with mutations at one or more residue positions 53, 62, 74, 75, 83, or any combination thereof, wherein said FVII mutant library comprises a plurality of FVII mutants that may be screened to select additional FVII mutants with increased biological activity.

As a further aspect of the invention, there is provided an inactivated factor VII/VIIa mutant or functional fragment thereof, comprising one or more mutation(s) in the epidermal growth factor-like (EGF-1) domain.

The mutation(s) in the inactivated FVII/FVIIa mutant may be at one, more than one, or all amino acid residues at positions 53, 62, 74, 75, 83 in the EGF-1 domain, or any combination thereof, such that the mutation(s) is/are to any amino acid residue that confers enhanced biological activity of said FVII/FVIIa. In an embodiment, the inactivated mutant FVII/FVIIa comprises one, more than one or all mutations selected from (S53N), (K62E), (K62D), (K62N), (K62Q), (K62T), (P74A), (A75D) and (T83K). In a preferred embodiment, the mutant FVII/FVIIa comprises the mutation (K62E) or mutation (K62T).

In another embodiment, the inactivated mutant FVII/FVIIa comprises an EGF-1 domain encoded by a nucleic acid sequence selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9 and SEQ ID NO:10, or any degenerate variant thereof. Alternatively, the inactivated mutant FVII/FVIIa may comprise an EGF-1 domain comprising a polypeptide sequence selected from SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19 and SEQ ID NO:20.

In an embodiment, the inactivated FVII/FVIIa mutant is inactivated by treatment with an irreversible active site inhibitor. Any irreversible active site inhibitor may be used, including peptide chloromethylketones. In an embodiment, a peptide chloromethylketone selected from FFR-ck, DEGR-ck, FPR-ck, PFR-ck and GGR-ck is used, and preferably FFR-ck or DEGR-ck is used as the active site inhibitor.

In further embodiments, there is provided a pharmaceutical composition comprising the inactivated FVII/FVIIa mutant described above, as well as a method for treating or preventing thrombosis, stroke, atherosclerosis, disseminated intravascular coagulation (DIC) or cancer, comprising administering a pharmaceutically effective amount of such composition.

For the purpose of the present invention, the following terms are defined below.

FVII/FVIIa shall refer to a product consisting of either the unactivated form (FVII) or the activated form (FVIIa) or mixtures thereof. FVII/FVIIa comprises proteins that have the amino acid sequence of native FVII/FVIIa, and includes proteins with slightly modified amino acid sequences, wherein such slight modifications may be in N-terminal amino acids or amino acid variations in the N-terminal region that do not affect FVIIa activity, and may also include naturally occurring allelic variations that may exist in native human FVII/FVIIa. Although distinctions have been made, FVII, FVIIa or FVII/FVIIa may be used interchangeably in the present disclosure.

Modified FVII/FVIIa shall refer to a biologically active molecule derived from FVII/FVIIa by the substitution of one or more amino acid residues. For the purpose of the this disclosure, modified FVII/FVIIa may also be identified as mutant FVII/FVIIa.

FVII(AxyB) or AxyB refers to mutant FVII/FVIIa comprising a point mutation from amino acid A (A) to amino acid B (B) at amino acid residue xy of FVII/FVIIa. For example, FVII(S53N) or S53N would accordingly refer to mutant FVII/FVIIa comprising a point mutation from Serine (S) to Asparagine (N) at amino acid 53 of FVII/FVIIa.

For convenient reference, the amino acid abbreviations commonly used in the art are summarized below:

| | | |
|---|---|---|
| | 3 letter | 1 letter |
| Amino Acid | abbreviation | abbreviation |

| | | |
|---|---|---|
| Alanine | Ala | A |
| Cysteine | Cys | C |
| Aspartate | Asp | D |
| Glutamate | Glu | E |
| Phenylalanine | Phe | F |
| Glycine | Gly | G |
| Histidine | His | H |
| Isoleucine | Ile | I |
| Lysine | Lys | K |
| Leucine | Leu | L |
| Methionine | Met | M |
| Asparagine | Asn | N |
| Proline | Pro | p |
| Glutamine | Gln | Q |
| Arginine | Arg | R |
| Serine | Ser | S |
| Threonine | Thr | T |
| Valine | Val | V |
| Tryptophan | Trp | W |
| Tyrosine | Tyr | Y |
| γ-carboxyglutamic acid | Gla | V |

Biological activity shall refer to a function or set of functions performed by a molecule in a biological context (i.e. in an organism or an in vitro facsimile). For the purpose of this disclosure, biological activity may refer to catalytic and effector activities. Biological activity may refer to binding affinity, which preferably refers to the binding of FVII/FVIIa to TF, or to clotting activity, which preferably refers to the ability to initiate the coagulation cascade.

### BRIEF DESCRIPTION OF THE DRAWINGS

Further features and advantages of the present invention will become apparent from the following detailed description, taken in combination with the appended drawings, in which:

FIG. 1 illustrates an alignment diagram of the EGF-1 domains of human FVII and rabbit FVII; the N-terminal amino acid residues 46-83 of FVII were aligned using the software program GENEPRO. The 5 non-conserved amino acid residues at positions 53, 62, 74, 75 and 83 are highlighted in bold.

FIG. 2. Transient expression of human rFVII mutant proteins. Human wild-type(WT) rFVII and the rFVII EGF1 domain mutant proteins S53N, K62E, P74A, A75D, T83K and rabEGF1 were transiently expressed in HEK293 cells. FVII antigen concentration was determined by human FVII-specific ELISA. Data are the means ±SEM, n≧3.

FIG. 3. Analysis of purified rFVIIa mutant proteins by SDS-PAGE. Electrophoretogram stained with coomassie blue. Lanes 1-6 contained a protein MW standard, plasma-derived FVIIa(ERL), wild-type rFVIIa(Novo Nordisk Inc., NN), rFVIIa(K62E), rFVIIa(A75D) and wild-type rFVIIa(Genentech Inc., Gen) respectively.

FIG. 4 represents the inhibition of binding of biotinylated plasma-derived FVII to full-length, relipidated, human TF in a competitive ELISA developed in our laboratory. The IC₅₀ for rFVII(K62E) was calculated to be 5-fold lower than for either plasma-derived or wild-type rFVII. Standard FVII=plasma-derived zymogen FVII (Enzyme Research Labs); Gentech FVII=wild-type zymogen rFVII (Genentech Inc.); K62E rFVII zymogen was purified in the laboratory of the inventors/applicants. The data illustrate the relative affinity of purified rFVII(K62E) mutant protein for TF via inhibition of binding of biotinylated plasma-derived zymogen FVII to full-length, relipidated human TF via competitive ELISA.

FIG. 5. Effect of FFRck and DEGRck on the affinity of rFVIIa(K62E) and rFVIIa(wt) for tissue factor (TF). The figure illustrates the inhibition of binding of biotinylated, zymogen FVII to full-length relipidated human TF by either rFVIIa or rFVIIa(K62E) or their derivatives in a competitive ELISA. Data are the mean molar ratios (IC₅₀, n=4) of the various inhibitors. The asterisks*** indicate a statistically significant difference from rFVIIa(wt)-FFR (P<0.001) using the Tukey-Kramer multiple comparisons t test.

FIG. 6. Relative prolongation of the clotting time of human plasma (A) and rabbit plasma (B) by rFVIIa(K62E)-FFR{□} versus rFVIIa(wt)-FFR{+} and pdFVIIa-FFR{o} in the presence of varying molar ratios of rFVIIai inhibitors. Data are the means of quadruplicate determinations.

It will be noted that throughout the appended drawings, like features are identified by like reference numerals.

### DETAILED DESCRIPTION OF THE INVENTION

The seminal role of the first epidermal growth factor-like (EGF1) domain of human factor VII (FVII) in binding to tissue factor (TF) has been established. The variable activity of TF from various animal species in initiating coagulation in heterologous plasma is well known. Increased coagulant activity of rabbit plasma (i.e. FVII) with human TF might be explained by the 5 non-conserved amino acids in the rabbit versus the human FVII EGF1 domain. Accordingly, using recombinant DNA methodology, we have “rabbitized” the human FVII EGF1 domain either by exchanging the entire EGF1 domain creating human FVII(rabEGF1) or by the single amino acid substitutions S53N, K62E, P74A, A75D, T83K, and other K62 mutations, such as K62D, K62N, K62Q, K62T. After transient expression in HEK293 cells, supernatant medium containing the unpurified, recombinant FVII (rFVII) mutant proteins were analyzed for coagulant activity, amidolytic activity and affinity of binding to full-length, relipidated human TF by competitive ELISA. Total rFVII mutant protein antigen secreted ranged from 18% to 135% of wild-type rFVII (112 ng/ml medium). Clotting activity of the unpurified rFVII mutant proteins was either depressed or unchanged. Amidolytic activity of the unpurified rFVII mutant proteins was not significantly different from wild-type rFVII. Notably, 3/6 unpurified rFVII mutant proteins had increased affinity for human TF in the rank order rFVII(rabEGF1) [3,3-fold]>rFVII(K62E) [2,9-fold]>rFVII(A75D) [1,7-fold]. To further validate these results a HEK293 cell line permanently expressing rFVII(K62E) was established and the mutant protein was purified to homogeneity from the serum-free culture medium by Q-Sepharose ion-exchange chromatography. Purified rFVII(K62E) had 1.9-fold greater clotting activity and 5-fold greater affinity for TF as compared to human rFVII(WT). The K_{D} of rFVII(k62E) for soluble human TF was 1.3 nM compared to 7.2 nM for rFVII(WT). We conclude that interspecies substitution of selected amino acid residues of the human FVII EGF1 domain confirms the primary role of the EGF1 domain in TF binding. This strategy facilitates the creation of mutants of human FVII with both enhanced biological activity and/or affinity for TF.

FVII, a 50 kDa glycoprotein of human plasma [1] is essential for the initiation of the clotting cascade in man[2, 3]. Recent evidence has supported a role for activated FVII(FVIIa) in TF-mediated signal transduction [4, 5], tumor angiogenesis and metastasis [6, 7] and the inflammatory response during disseminated intravascular coagulation [8]. In the quest for new anticoagulant and anti-inflammatory drugs, a number of strategies have been explored in attempts to inhibit the FVII-TF interaction. This is a formidable biological task as both zymogen FVII and FVIIa bind with high affinity to soluble human TF with a K_{D} of 7.5 nM and 5.1 nM respectively [9]. New, potentially important anticoagulants include humanized monoclonal antibodies to TF [10] and variants of human soluble TF [11]. A different approach has resulted in the description of inhibitory peptides to exosites in the heavy chain of human FVII [12, 13]. To date studies of both natural [14-6] and site-directed mutants of FVII [17] have universally described FVII mutant proteins with decreased affinity for TF. The impetus for this work was an early observation of Janson et al. [18] who described a 4-fold increased clotting activity of rabbit plasma (i.e FVII) versus human plasma (FVII) on incubation with human TF. Since 43% of the contact area of TF with FVII lies within the FVII EGF1 domain [19] we believe, and have shown, that the difference in FVII clotting activities noted above might reside in the 5 amino acid residues which differ between the rabbit [20] and human FVII EGF1 domains. Accordingly, we have both substituted the entire rabbit EGF1 domain and the corresponding rabbit amino acid for its human counterpart at each of these 5 residues. This has resulted in the creation of human FVII mutant proteins with enhanced affinity for human TF and increased enzymatic activity.

The present invention is directed to mutants of FVII EGF-1 domain. More specifically, the present invention is directed towards mutants of human FVII EGF-1 which increase biological activity of FVII/FVIIa, and more preferably the affinity of FVII/FVIIa for TF, and the resulting enhanced coagulation activity.

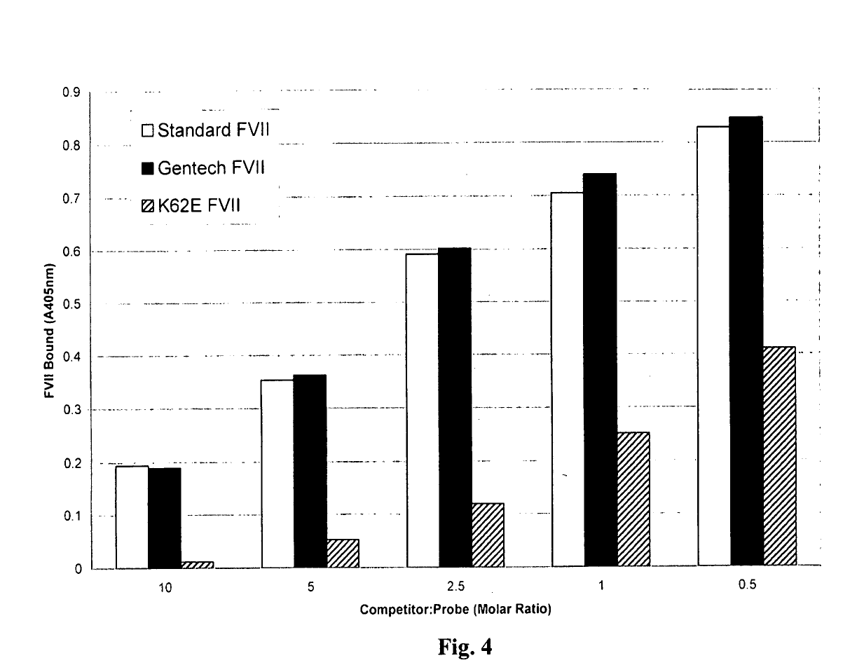

In accordance with preferred embodiment of the present invention, there is provided a mutant of human recombinant factor VII(K62x), wherein x is preferably E, i.e mutant FVII(K62E), or any other mutant with improved biological activity, such as mutant K62T, i.e mutant FVII(K62T). In addition, a cell line permanently expressing rFVII(K62E), or any of the other rFVII mutants exhibiting improved affinity, has been established in accordance with the present invention. The recombinant protein, namely rFVIIa(K62E), has been purified to homogeneity in milligram quantities. Results with purified rFVIIa(K62E) indicate that it has at least a 5-fold greater affinity for human TF than does wild-type rFVIIa, as determined by competitive ELISA (please refer to FIG. 4). A subsequent blinded experiment, confirmed the 5-fold increased affinity of rFVIIa(K62E) for TF, where the confirmatory blinded experiment was performed independently by surface plasmon resonance technology. Quantitation of the coagulant activity of rFVIIa(K62E) by prothrombin time (PT) assay have indicated that it has 1.5 to 2-fold enhanced enzymatic activity in vitro (as identified in Table 2 below).

The developments of the present invention are especially useful, and comprise scientific and clinical significance. More specifically, recombinant human FVIIa (NovoSeven from Novo Nordisk), i.e. a purified commercial wild-type fFVIIa, is currently being used clinically in hemophilia A patients with antibody to factor VIII [39] but the amounts required are high due to the competition of patient zymogen plasma FVII with rFVIIa for available TF [45]. Artificially inactivated human rFVIIa, called rFVIIai, has been shown to effectively inhibit thrombosis and death in a baboon model of DIC [8]. Both of these potential uses of human rFVIIa would be greatly facilitated by the advent of mutants of FVII, such as the mutants provided in the present invention, with either increased enzymatic activity (in hemophilia) or increased affinity for TF (a better competitive inhibitor) for thrombosis associated with disseminated intravascular coagulation(DIC), atherosclerosis and cancer. Moreover, as noted above, mutants of human rFVIIa with increased affinity for TF and/or clotting activity could make the current use of wild-type rFVIIa obsolete. Furthermore, enzymatically-inactive mutant forms of rFVIIa with high-affinity for TF could become novel anticoagulants.

The species-specific FVII-TF interaction, i.e. FVII from rabbits and mice both exhibited dramatically increased enzymatic activity with human TF rather than with homologous TF, and the allosteric interaction(s) between the FVII EGF-1 and protease domains modulated both TF binding and the enzymatic activity of FVII were noted to be of significance. This evidence sequentially precipitated the exchanging of 5 amino acids of the human FVII EGF-1 domain for those of rabbit FVII [20] using site-directed mutagenesis technology [15]. In accordance with an embodiment of the present invention, point amino acid mutations made were S53N, K62E, K62D, K62N, K62Q, K62T, P74A, A75D, T83K i.e. the human FVII EGF-1 domain was “rabbitized” at each of these non-conserved amino acid residues resulting in the creation of unique variants of human FVII. Each of the recombinant human FVII chimeric cDNAs were then transfected into human kidney 293(HEK) cells and transient expression of the FVII chimeric proteins at levels of 20-120 ng FVII antigen/ml culture media was observed. Preliminary characterization of the above unpurified, FVII mutant proteins indicated that several of the mutant proteins, and in particular FVII(K62E) had increased affinity for TF.

TABLE 1
Coagulant Activity of Purified Recombinant FVII (K62E).

| FVII | Clotting Time | Clotting Activity |
|---|---|---|
| Preparation | (Sec.) | (U/mg) |
| wt-rFVII-Genentech | 21.7 ± 0.6 | 2370 ± 120 |
| wt-FVII-ERL | 28.4 ± 0.6 | 1170 ± 20 |
| rFVII (A75D) | 22.5 ± 1.7 | 2140 ± 190 |
| rFVII (K62E) | 17.2 ± 0.5** | 3850 ± 390** |
| Legend: Purified commercial wild-type (wt) rFVII was from Genentech Inc. Plasma-derived human FVII was from Enzyme Research Labs Inc. (ERL). rFVII (A75D) and rFVII (K62E) were purified from serum-free HEK293 cell culture medium by Q-Sepharose ion-exchange chromatography. A conventional prothrombin time (PT) clotting assay was performed using FVII-depleted pooled human plasma and recombinant human, relipidated TF. The asterisks** indicate a highly significant difference between the clotting activities | | |
| # of rFVII (K62E) and rFVII-Genentech (p ≦ 0.01, n = 3). Statistical analysis was by ANOVA using the InStat3 software package. | | |

Subsequently, both humanized monoclonal antibodies to human TF and several variants of human TF have been described [10]. Research with human FVII has resulted in the description of inhibitory peptides to several exosites in human FVII [46] as well as mutants of the FVII heavy chain with increased intrinsic enzymatic activity [38].

To date, studies of both natural [16] and synthetic mutants of FVII [17] have universally described FVII molecules with decreased enzymatic activity and affinity for TF. In contrast, the earlier work of Janson et al. [18] described 4-fold increased clotting activity of rabbit plasma FVII versus human plasma FVII with human TF. Since 43% of the contact area of TF with FVII lies within the FVII EGF-1 domain [19] we postulated that the difference in FVII clotting activities noted above might reside in the 5 amino acid residues which differ in the rabbit [20] and human FVII EGF-1 domains.

Accordingly, individual, and combinatorial, substitution within the human FVII EGF-1 domain, and more specifically, at one, more or all these 5 amino acid positions for the corresponding rabbit amino acid has resulted in the creation of ‘rabbitized’ human FVII mutant proteins with enhanced affinity for human TF and increased enzymatic activity.

Production of FVII/FVIIa Mutants

Experimental Procedures

Reagents Human FVII cDNA was subcloned in the EcoRI-HindIII site of the expression vector pCMV5 as described [15]. Cloning vector pUC19 DNA was from New England Biolabs (Beverly, Mass.). Plasmid DNA was amplified in Escherichia coli XL-1 Blue (Stratagene, La Jolla, Calif.). Oligonucleotide synthesis and automated DNA sequence analysis were performed in the molecular biology facility MOBIX, McMaster University. Dulbecco's Modified Eagles (DMEM)-Ham's F12 media was from Sigma-Aldrich Co. (St. Louis, Mo.). The tissue culture medium supplement bovine albumin-insulin-transferrin (BIT 9500) was from Stem Cell Technologies (Vancouver, BC).

Site-Directed Mutagenesis. Oligonucleotide site-directed mutagenesis (Clontech, Palo Alto, Calif.) was performed on the FVII EGF1 domain in the vector pUC19 as previously described [15]. The mutagenic primers employed were:

| | |
|---|---|
| S53→N | (5′-GTGTGCCTCAAACCCATGCCAGAATG-3′), |
| K62→E | (5′-GGGCTCCTGCGAGGACCAGCTC-3′), |
| P74→A | (5′-GCTTCTGCCTCGCTGCCTTCGAG-3′), |
| A75→D | (5′-CTGCCTCCCTGACTTCGAGGGC-3′), |
| T83→K | (5′-GCCGGAAGTGTGAGAAACACAAGGATGACC-3′), |
| K62→D | (5′-GGGCTCCTGCGACGACCAGCTC-3′), |
| K62→N | (5′-GGGCTCCTGCAACGACCAGCTC-3′), |
| K62→Q | (5′-GGGCTCCTGCCAGGACCAGCTC-3′), |
| and | |
| K62→T | (5′-GGGCTCCTGCACGGACCAGCTC-3′). |

Human rFVII with a rabbit EGF1 domain i.e. rFVII(rabEGF1) was created by site-directed mutagenesis using the unique restriction sites BstEII and NsiI at the 5′ and 3′ ends of the human FVII EGF1 domain respectively. The BstEII site was generated using the primer:

5′-CTTACAGTGATGGTGACCAGTGTGCCTC-3′.

This base substitution did not change the amino acid sequence of human FVII. The NsiI site was generated using the primer:

5′-CGGAACTGTGAGATGCATAAGGATGACCAGC-3′.

Creation of the new NsiI restriction site also altered the amino acid sequence of human FVII changing residue T83→M. After excision of the human FVII EGF1 domain DNA by BstEII-NsiI restriction endonuclease digestion and subcloning of the rabbit FVII EGF1 domain DNA in its place, the codon at position 83 was corrected from M83→K by site-directed mutagenesis using the primer:

5′-GGTCGCAACTGTGAGAAACACAAGGATGACCAGC-3′.

Rabbit FVII EGF1 domain DNA was prepared using rabbit FVII template cDNA [20] by a standard polymerase chain reaction utilizing the forward primer:

5′-TACAATGATGGTGACCAGTGTGCCTCC-3′,

and the reverse primer:

5′-TCTTATGCATCTCACAGTTGCGACCCTCG-3′.

The fidelity of all FVII mutant DNAs were confirmed by automated DNA sequence analysis.

Mammalian Cell Culture and Transient Expression of rFVII Mutant Proteins. Wild-type and mutant FVII cDNAs in the vector pCMV5 were transfected into HEK293 cells using Lipofectin reagent (InVitrogen Corp., San Diego, Calif.) as previously described [22]. HEK293 cells were routinely maintained in DMEM-F12 medium supplemented with 10% fetal calf serum, 100 U/ml penicillin-streptomycin and 100 ng/ml vitamin K. HEK293 cell conditioned media were collected for analysis 72 hr post-transfection and concentrated 6-fold by Amicon ultrafiltration prior to quantitation by FVII-specific ELISA.

Permanent Expression of rFVII Mutant Proteins. Two HEK293 cell lines permanently expressing recombinant FVII (rFVII) mutants FVII(A75D) and FVII(K62E) established essentially as described [20]. Briefly, both FVII mutant cDNAs were subcloned into the EcoRI-HindIII site of the expression vector pCMV5 and co-transfected into HEK293 cells with the selection plasmid pSV2neo. After 2-3 weeks post-transfection, G418-resistant clones were assayed for synthesis of human FVII by ELISA. Optimal FVII-synthesizing cell clones were expanded into NUNC triple-flask cell factories and the supernatant medium was collected weekly. Purification of rFVII from HEK293 cell conditioned medium was greatly facilitated by the use of serum-free, phenol red-free DMEM-F12 supplemented with 1 mg/ml bovine serum albumin, 1 μg/ml bovine insulin, 20 μg/ml human transferring (BIT), 100 ng/ml vitamin K and penicillin-streptomycin. Confluent HEK293 cells remained adherent to the plastic substratum and continued to synthesize rFVII normally for 3-4 weeks in the above medium.

Purification of rFVII Mutant Proteins. Recombinant FVII mutant proteins were purified from serum-free HEK293 conditioned medium using a modification of the Q-Sepharose pseudoaffinity chromatography technique [23]. Briefly, HEK293 cell serum-free conditioned medium was collected and filtered through one layer of Whatman No. 1 filter paper. Benzamidine and Na₂EDTA were added to final concentrations of 10 mM and 5 mM respectively. The medium was stored frozen at −40° C. One liter of HEK293 cell conditioned medium was concentrated to 250 ml using a Millipore pump and PLTK prep-scale TFF cartridge (30,000 kDa molecular weight cut-off). The 250 ml concentrate was dialyzed overnight against 20 mM Tris, pH 8.0, 50 mM NaCl, 0.05% azide, 1 mM benzamidine, 1 mM EDTA at 4° C. The dialyzed sample was readjusted to 10 mM benzamidine and 5 mM EDTA. Conductivity of the dialyzed sample was routinely less than 10 μmhos. If not, distilled water was added. Q-Sepharose fast flow (1.5×25 cm, bed volume 50 ml) was equilibrated with 3 column volumes of 20 mM Tris pH 8.0, 50 mM NaCl, 10 mM benzamidine, 5 mM EDTA. All subsequent chromatographic steps were at 4° C. The dialyzed, concentrated medium was applied to the column at a flow rate of 2 ml per min. The column was then washed with 5 column volumes of equilibration buffer followed by 5 column volumes of equilibration buffer without EDTA. rFVII was eluted from the column with 250 ml of equilibration buffer without EDTA containing 10 mM CaCl₂. The majority of rFVII eluted in the first 150 ml. Eluted rFVII was concentrated to 5 ml by Amicon ultrafiltration. The purity of the starting rFVII concentrate and the eluted protein were analyzed by SDS-PAGE and Western blot analysis using biotinylated, monospecific sheep anti-FVII IgG. A second passage over Q-Sepharose was needed to achieve 95%+pure material. For the second stage, a column of 4-5 ml bed volume, applying maximum 20-25 mg total protein per ml gel was used. Once again protein was bound using the low ionic strength equilibration buffer but eluted with 5 mM CaCl₂. In some purifications final removal of albumin from FVII K62E, or other FVII mutant, was accomplished using sheep anti-BSA IgG coupled to sepharose.

Quantitation of FVII, FVIIa and Total Protein. Total rFVII/rFVIIa antigen levels were determined by solid-phase enzyme-linked immunoabsorbent assay (ELISA) as previously described [20]. Briefly, the assay incorporated monospecific polyclonal sheep anti-human FVII IgG as the trapping antibody and biotinylated monospecific polyclonal sheep anti-human FVII IgG as the detecting antibody. Biotinylated antibody binding was quantitated using streptavidin-alkaline phosphatase and the enzyme substrate PNPP. Either purified plasma-derived human FVII or purified human rFVII were used to generate a standard curve. Data were plotted as the absorbance at 405 nm versus the FVII antigen concentration. The assay was linear in the range 1-25 ng/ml FVII antigen. Total protein concentrations were determined either by BCA assay (Pierce Scientific Co., Rockford Ill.) or the Bradford coomassie blue reagent (Sigma-Aldrich, St. Louis, Mo.).

Coagulant and Amidolytic Activity of rFVII Mutant Proteins. Coagulant activity of the various FVII samples was measured by prothrombin time(PT) assay using FVII-depleted human plasma and relipidated full-length human thromboplastin as previously described [24]. Amidolytic activity of rFVIIa with the chromogenic peptide substrate S-2222 was determined as described [24].

Determination of the Relative Affinity of rFVII Mutant Proteins for TF by Competitive ELISA. The binding of biotinylated, plasma-derived FVII to relipidated, full-length rTF and the quantitation of the relative affinity of rFVII mutant proteins for rTF by inhibition of biotinylated FVII binding (IC₅₀) has been described in detail elsewhere [24].

Determination of the Absolute Affinity of rFVII Mutant Protein for sTF by Surface Plasmon Resonance. An anti-TF antibody was immobilized on the BIAcore flow cell at high density and subsequently reacted with recombinant sTF. Dilutions of wild-type or mutant FVII molecules were then injected into the BIAcore flow cell and binding kinetics were determined. Data from reference cells containing the same amount of anti-TF antibody but no sTF were subtracted to correct for non-specific binding. The reference cell contained the same amount of anti-TF antibody but no TF [25].

Statistical Analysis. Linear regression analysis, Student's t test, analysis of variance and standard error of the mean were performed using the InStat 3.05 software package for Windows 98 (GraphPad Software, San Diego, Calif.).

Results

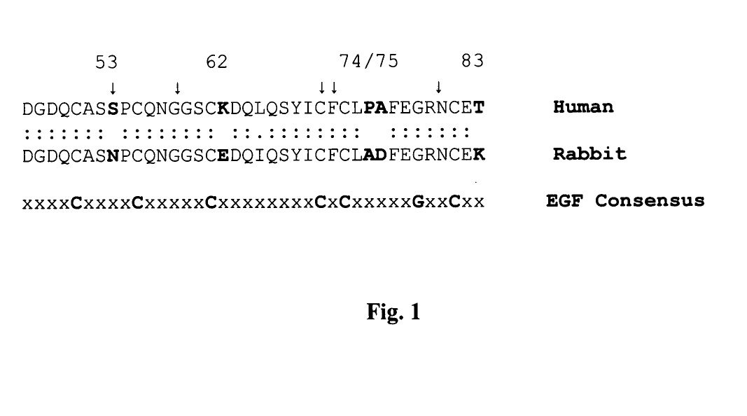

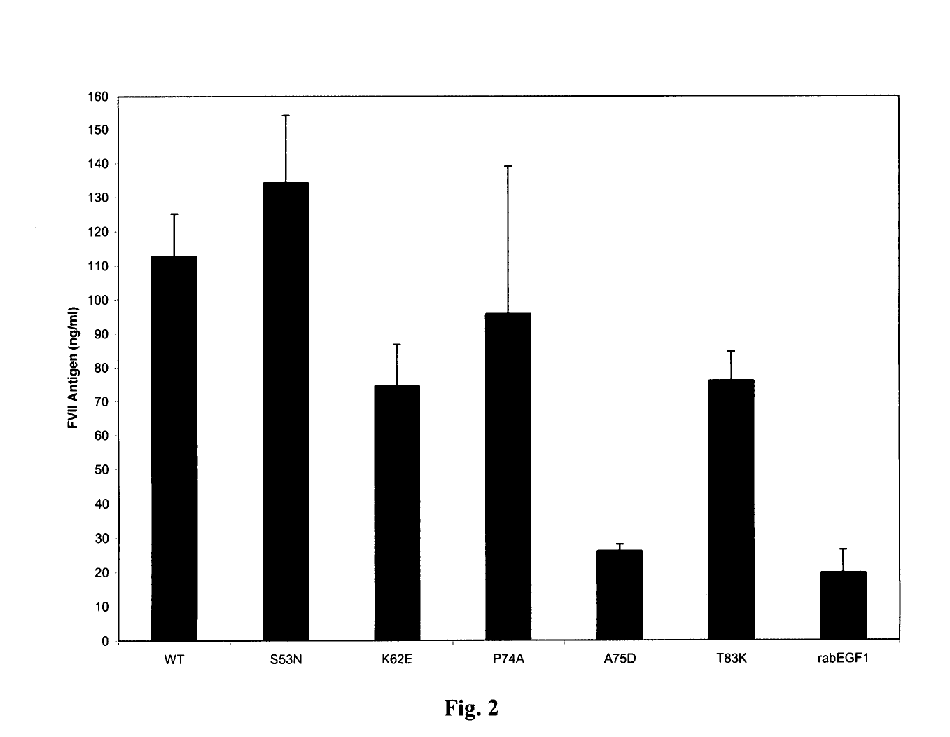

DNA mutagenesis, transient expression and characterization of unpurified human rFVII EGF-1 mutant proteins. Alignment of the 38 amino acids of the human and rabbit FVII EGF-1 domains (FIG. 1) illustrates that there are 5 non-conserved amino acids at residues 53{S→N}, 62{K→E}, 74{P→A}, 75{A→D} and 83{T→K}. Using site-directed DNA mutagenesis, the human FVII molecule was “rabbitized” at each of these amino acid residues to create rFVII(S53N), rFVII (K62E), rFVII (P74A), rFVII (A75D) and rFVII (T83K). In addition, it should also be noted, that in addition to mutations that ‘rabbitized’ the FVII EGF-1, additional mutations, such as mutations K62D, K62N, K62Q, K62T are also provided in the present invention. Nevertheless, the collective effect of all five amino acid changes was examined by restriction endonuclease excision of the human FVII EGF1 domain DNA and substitution of the PCR-generated rabbit FVII EGF1 DNA to create rFVII(rabEGF1). Fidelity of the full-length mutant rFVII cDNA sequences was confirmed by automated DNA sequence analysis. The mutant rFVII cDNAs were then excised from pUC19 via EcoRI-HindIII endonuclease digestion and subcloned into the mammalian expression vector pCMV5. Three days after liposome-mediated pCMV5(rFVII) DNA transfection into HEK293 cells, transient expression of wild-type and mutant rFVII molecules was quantitated by FVII-specific ELISA using polyclonal anti-human FVII IgG. FIG. 2 represents the mean rFVII antigen expression observed for each of the 6 mutant human rFVII proteins as compared to wild-type (WT) human rFVII. Generally, rFVII mutant proteins and rFVII(WT) were expressed at levels between 70-130 ng rFVII/ml culture medium, with the exception of rFVII(A75D) and rFVII(rabEGF1) which were expressed/secreted at significantly at lower levels. The biological activity of the transiently-expressed, unpurified rFVII mutant proteins were then compared to rFVII (WT). Specific activities of the rFVII mutant proteins for either the peptidyl substrate S-2222 (amidolytic assay) or a macromolecular substrate (PT assay) were not statistically different from wild-type rFVII (data not shown). However, multiple determinations of the affinity of each of the mutant rFVII proteins for immobilized human TF by competitive ELISA (Table 2) indicated that transiently expressed rFVII(K62E) and rFVII(rabEGF1) bound at least 2.9-fold and 3.3-fold more tightly than did rFVII (WT). Other rFVII EGF-1 mutant proteins were either unchanged or decreased in their affinity for TF. In some experiments, the mutant protein rFVII(A75D) appeared to have enhanced affinity for TF but the data obtained by repeated competitive ELISA binding did not achieve statistical significance.

It should be noted that although mutants(S53N), (K62E), (P74A), (A75D), or (T83K), comprising individual amino acid mutations are detailed in this text, other modified FVII/FVIIa according to the present invention are also included herein, and may additionally exhibit increased biological activities than the mutant presently detailed herein.

That is to say, the present invention contemplates all mutations at one or more, or any combination of mutations at positions 53, 62, 74, 75 and 83 of the EGF-1 domain of FVII, wherein the mutations embodied in the present invention exhibit improved biological activity. For example, mutation K62E, or K62T, or A75D, or any other single or multiple point mutation embodied in the present invention showing improved biological activity is embodied herein. Such improved biological activity may comprise an increase in binding affinity for TF, improved anti-coagulant or anti-inflammatory activities.

Where, in a preferred embodiment of the present invention, other FVII EGF1 domain mutants displaying increased biological activities where K62 rFVII EGF1 mutants. Accordingly, all rFVII EGF1 domain mutants exhibiting improved biological activities, such as increased clotting activity, are embodied in the preset invention. Where, more specifically, mutants K62D, K62E, K62N, K62Q, and K62T have been shown to exhibit increased clotting activity, where mutant K62T showed the most improved increase in clotting activity, where clotting activity was increased 2.3-fold compared to the wild-type. Table 2 below summarizes the relative FVII coagulant activity for some rFVII EGF1 K62 mutants, wherein all mutants summarized exhibit increased clotting activity with respect to the wild-type FVII.

TABLE 2
FVII K62 mutant coagulant activity relative to WT

| FVII | Coagulant | Relative |
|---|---|---|
| Mutant | Activity (U/mg) | Activity |
| WT | 2085 ± 520 | 1.0 |
| K62D | 3025 ± 365 | 1.4 |
| K62E | 3490 ± 610 | 1.7 |
| K62N | 2800 ± 450 | 1.3 |
| K62Q | 2955 ± 540 | 1.4 |
| K62T | 4835* ± 730 | 2.3 |

The data show the relative FVII coagulant activity ± SEM (n = 11) of unpurified FVII proteins mutated at the K62 amino acid residue. The asterisk* represents statistical difference (p ≦ 0.05) as compared to wild-type factor VII [FVII (WT)].

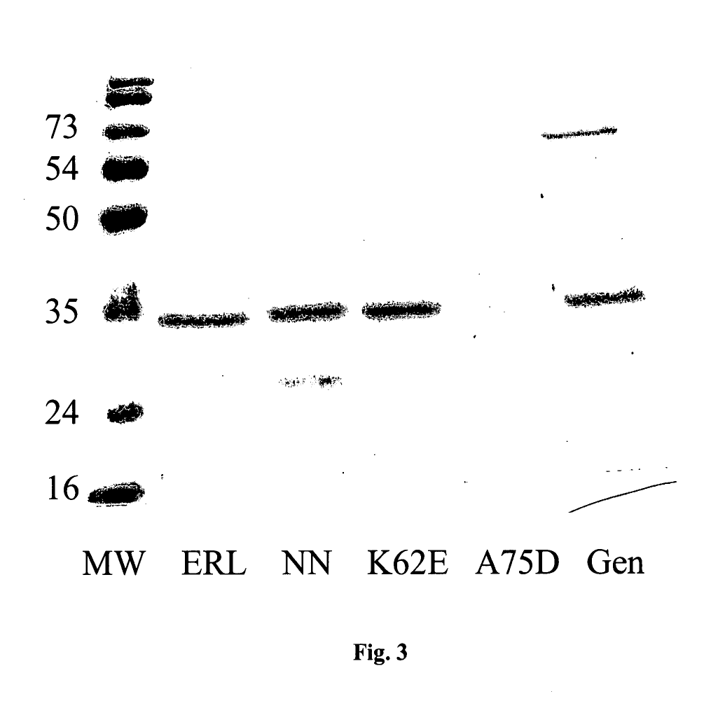

Purification of rFVII(K62E) and rFVII(A75D). To confirm the above results, both rFVII(K62E) and rFVII(A75D) were purified to homogeneity from 1-2 liters of HEK293 serum-free conditioned medium. Although both mutant rFVII proteins were ≧99% in the zymogen form in unprocessed conditioned medium, initial purification of the mutant proteins via pseudoaffinity chromatography on Q-Sepharose ion-exchange resin resulted in partial activation of mutant rFVII to the rFVIIa form. In order to facilitate comparison of the various rFVII molecules, autoactivation of both rFVII(K62E) and rFVII(A75D) to their activated forms was allowed to occur by purposely omitting benzamidine from the column chromatography buffers. The purified rFVIIa EGF-1 mutant proteins were compared to commercially available plasma-derived FVIIa, rFVIIa synthesized in BHK cells and rFVIIa synthesized in HEK293 cells. Analysis by SDS-PAGE and coomassie blue staining (FIG. 3) revealed that all FVII preparations were essentially fully activated to FVIIa. A high molecular weight protein contaminant of wild-type rFVIIa (Genentech Inc) can be seen in lane 6. All FVIIa preparations exhibited the expected heavy chain at ˜30 kDa and light chain at ˜20 kDa. Western blot analysis of the above gel with human FVII-specific polyclonal IgG revealed virtually identical isoforms of the rFVIIa light chain in all preparations.

Functional Analysis of Purified rFVII (K62E). To confirm and extend the above results rFVII(K62E) was permanently expressed in HEK293 cells and purified to homogeneity from 1-2 liters of HEK293 serum-free conditioned medium. After purification via pseudoaffinity chromatography on Q-Sepharose ion-exchange resin rFVII(K62E) was >95% pure as judged by SDS-PAGE with coomassie blue staining and western blot analysis (data not shown). Purification of rFVII(K62E) resulted in 10-20% activation of rFVII(K62E) to rFVIIa(K62E). As seen in FIG. 4 the relative affinity of purified rFVII(K62E) for full-length relipidated TF as assayed by competitive ELISA was 5-fold greater than either human plasma-derived FVII or human rFVII(WT). This result was confirmed independently by surface plasmon resonance experiments (Table 7). The K_{D} of rFVII(K62E) for human sTF was 1.3 nM, i.e. 5-fold lower than rFVII(WT) and greater than 10-fold lower than pdFVII. In addition to its enhanced affinity for TF, rFVII(K62E) exhibited a 1.9 fold increase in coagulant activity (Table 7) as compared to rFVII(WT).

TABLE 3
A comparison of the biological activity of purified
rFVIIa (K62E) and rFVIIa (A75D) with wild-type FVIIa.

| FVII | Clotting | | Affinity |
|---|---|---|---|
| Preparation | Activity | IC₅₀ | Increase* |
| wt-rFVIIa¹ | Unchanged | 0.61 | 1.8X |
| Plasma | Unchanged | 1.14 | 1.0X |
| FVIIa | | | |
| rFVIIa K62E | Increased | 0.16 | 7.1X |
| rFVIIa | Increased | 0.19 | 6.0X |
| A75D | | | |

wt-rFVII¹ purified commercial wild-type (wt) rFVIIa from Novo Nordisk Inc.
wt-FVII² plasma-derived human FVII from Enzyme Research Labs
*affinity increase of binding betwaan rFVII mutant and human TF, as determined by competitive ELISA (as described in [24])

TABLE 4
Summary of Activity Changes of Unpurified
Recombinant FVII mutants.

| FVII | Clotting³ | amidolytic⁴ | binding |
|---|---|---|---|
| Preparation | activity | activity | affinity⁵ |
| wt-rFVII¹ | 1.0 | 1.0 | 1.0X |
| rFVII S53N | Depressed | Unchanged | 0.4X |
| rFVII K62E | Unchanged | Unchanged | 2.3X |
| rFVII P74A | Unchanged | Unchanged | 1.5X |
| rFVII A75D | Increased | Unchanged | 1.6X |
| rFVII T83K | Unchanged | Unchanged | 1.2X |

wt-rFVII¹ unpurified wild-type (wt) rFVII from our laboratory.
wt-FVII² plasma-derived human FVII from Enzyme Research Labs
clotting activity³ (as described in [24])
clotting activity³ (as described in [24])
amidolytic activity⁴ (as described in [24])
binding affinity⁵ of recombinant FVII mutants to full-length, relipidated human TF, relative to wt-rFVII, as determined by competitive ELISA (as described in [24])

Table 5 summarizes the changes in FVII mutant Affinities for human TF.

TABLE 5
Binding of transiently expressed rFVII mutant
proteins to TF.

| FVII | Relative Affinity | Relative Increase |
|---|---|---|
| Mutant | for TF (IC₅₀) | In Affinity for TF |
| WT | 2.0 ± 0.5 | 1.0 |
| S53N | 5.3 ± 2.0 | 0.4 |
| K62E | 0.7 ± 0.2* | 2.9 |
| P74A | 2.2 ± 1.0 | 0.9 |
| A75D | 1.2 ± 0.5 | 1.7 |
| T83K | 1.9 ± 0.2 | 1.1 |
| rabEGF1 | 0.6 ± 0.3* | 3.3 |

The data show the normalized competitive ELISA IC₅₀ values (ng FVII/ml) for inhibition of biotinylated plasma FVII binding to relipidated, full-length human TF. Data are the means ± SEM, n ≧ 3.
The asterisk * represents p ≦ 0.05 as compared to the mean of FVII (WT).

TABLE 6
Absolute affinity of purified rFVII (K62E) for sTF
determined by surface plasmon resonance.

| FVII Sample | kₐ × 10⁻⁵ (M⁻¹ S⁻¹) | k_{d} × 10³ (s⁻¹) | K_{D} (nM) |
|---|---|---|---|
| rFVII (WT) | 4.3 ± 0.5 | 3.1 ± 0.1 | 7.2 ± 1.1 |
| pdFVII | 1.5 | 4.4 | 29.0 |
| rFVII (K62E) | 26.0 | 3.4 | 1.3 |

Purified rFVII (WT) was from Genentech Inc. Data are the means ± SEM.

TABLE 7
Coagulant Activity of Purified rFVII (K62E)

| FVII | Clotting Activity | Relative Increase In |
|---|---|---|
| Sample | (U/mg) | Coagulant Activity |
| rFVII (WT) | 7580 ± 575 | 1 |
| pdFVII | 2240 ± 90 | 0.3 |
| rFVII (K62E) | 14275 ± 395** | 1.9 |

Purified rFVII (WT) was from Genentech Inc. Data are the means ± SEM, n = 4. The asterisks ** indicate a statistically significant difference between rFVII (K62E) and rFVII (WT), p ≦ 0.01.

Discussion

The first epidermal growth factor-like domain of human coagulation FVII is essential for high-affinity binding to its cell surface receptor TF [14, 19, 26, 27]. In some studies, replacement of the entire human FVII EGF1 domain with the homologous rabbit FVII EGF1 region resulted in the formation of a chimeric human rFVII(rabEGF1) molecule which was poorly secreted from HEK293 cells (FIG. 2) but, in the unpurified form, exhibited greater than a 3-fold increase in affinity for TF (Table 5) and an approximate 2-fold increase in specific clotting activity with human TF relative to pdFVII (data not shown). Subsequently, we examined the effects of the 5 amino acid differences between the human and rabbit FVII EGF1 domains (FIG. 1) in isolation. Transient expression of the rFVII mutants in HEK293 cells revealed that rFVII(K62E) demonstrated a statistically significant increase in affinity for human TF (Table 5). Permanent expression and purification of the rFVII(K62E) mutant protein confirmed that it possessed approximately 5-fold increased affinity for human TF (FIG. 3, Table 6) and a 1.9-fold elevation in clotting activity (Table 7) relative to human rFVII(WT). This data confirms and extends the observations of Janson et al. [18], our previous results [28] and the results of others [29] who noted significant differences in the affinity and/or activity of human and rabbit FVII for homologous versus non-homologous TF.

Our laboratory has previously analyzed two naturally-occurring mutations of the FVII EGF1 domain which affect binding to TF. Both mutants rFVII(N57D)[15] and rFVII(R79Q) [14, 30] exhibited a 5-10 fold decrease in TF binding but the mechanisms of the two defects differed. Amino acid residue R79 of FVII has been shown to form both hydrophobic and hydrogen bonds with TF [19] whereas the mutation FVII(N57D) did not directly alter FVII contact with TF but caused a mis-folding of the FVII EGF1 domain [15]. rFVII(R79Q) mutant protein bound normally to the EGF1 conformation-sensitive monoclonal antibody 231-7 but rFVII(N57D) mutant protein did not [15]. Notably, the increased affinity of rFVII(K62E) protein for TF is associated with enhanced binding of rFVII(K62E) protein to monoclonal antibody 231-7 (data not shown), suggesting that the K→E substitution at position 62 has resulted in a conformational change in the rFVII(K62E) EGF1 domain. Although K62 is far removed from the FVII-TF interface [19], the K→E substitution potentially affects its neighboring amino acid residues D63, Q64, 169, C70 and F71 all of which either directly contact TF and/or act as ligands for the single Ca⁺⁺ bound within the FVII EGF1 domain.

Naturally-occurring mutants of the EGF1 domain have not been reported for either FVII or factor IX at the K62/K63 amino acid residue respectively. Furthermore, the C-K-D tripeptide sequence is absolutely conserved in the factor IX EGF1 domain whereas FVII EGF1 amino acid residue 62 is variously D (chicken), E (rabbit & bovine), Q (mouse & rat) and T (zebrafish) [20, 31, 32].

A number of laboratories have provided evidence for reciprocal allosteric interaction(s) between the EGF1 and protease domains of FVII [33-36]. Perturbations of the FVII EGF1 domain including monoclonal antibody 231-7 binding [33] and site-directed mutagenesis affecting the high-affinity Ca++binding site [34] have been shown to either enhance or decrease FVII catalytic activity respectively. We would therefore suggest that the K62→E amino acid substitution enhances rFVII coagulant activity via an allosteric effect on the protease domain [33]. Furthermore, we submit that the K62→E amino acid exchange likely causes an increased affinity for TF by altering either the conformation of the EGF1 domain alone or the relative orientation of the neighboring Gla and EGF1 domains [33, 34]. Our results are an interesting contrast to the recent data of Persson et al. [37, 38] who have demonstrated that mutagenesis of selected amino acid residues in the protease domain of rFVII can substantially increase the intrinsic activity of rFVII in an essentially TF-independent manner.

Recombinant FVIIa is currently approved for use in Canada in hemophilia A patients with acquired inhibitors to factor VIII. The chemical amounts of rFVIIa required is a minimum of 2-3 bolus injections of 90 ug rFVII/kg body weight [39] due to the competition of patient plasma FVII zymogen with the infused rFVIIa for available TF [40]. Thus rFVIIa currently constitutes the second most costly drug purchased by the Canadian Blood Services, Canada's blood agency. Conversely, chemically inactivated rFVIIa, termed rFVIIai, which exhibits increased affinity for TF in vitro [41], has been shown to be an effective inhibitor of thrombosis in vivo in a baboon model [8] and to enhance apoptosis of tumor cells in vitro [42-44]. Use of human rFVIIa/rFVIIai would be greatly facilitated by the advent of mutants of rFVIIa with either increased enzymatic activity (in hemophilia) or increased affinity for TF (a better competitive inhibitor) in patients with thrombosis and cancer. Accordingly, mutants of the present invention, wherein the preferred mutants of the present invention exhibit increased affinity for TF, and/or improved biological activity, may be used to enhance hemostasis in both hemophilia patients with circulating inhibitors and patients with thrombocytopenia, and may additionally be used as anti-coagulant and/or anti-inflammatory compounds for the treatment and/or prophylaxis of patients with thrombosis, disseminated intravascular coagulation or cancer.

### EXAMPLE

Pharmaceutical Preparations and Methods of Use

Compositions effective for modulating the biological activity of FVII/FVIIa in a mammal, and preferably a human, are encompassed in the present invention. A pharmaceutical composition of the present invention may comprise at least one modified factor FVII/FVIIa (mutant FVII/FVIIa) as herein described, or complexes or fragments thereof. According to a preferred embodiment, a pharmaceutical composition of the present invention comprises a modified form of factor FVII/FVIIa (mutant FVII/FVIIa) having a mutation at residue 62 thereof, such as mutation K62E. Preferably, a composition of the present invention improves FVII/FVIIa binding of TF and improves blood coagulation. Accordingly, a pharmaceutical composition of the present invention comprises a purified or unpurified protein, peptide, polypeptide, or functional fragment thereof for mutant FVII/FVIIa (K62E), or any mutant embodied herein having improved biological activity, or a nucleotide sequence or fragment thereof, in a vehicle (vector, cell, compound, vesicle) capable of providing and/or expressing mutant FVII/FVIIa in, or to, said patient.

The invention provides modulators (e.g., effectors) of FVII/FVIIa activity and their therapeutic administration. These compounds include one or more of the FVII/FVIIa mutants prepared and/or identified by the methods of the invention. A compound that can be used therapeutically also includes a purified polypeptide, immunogenic polypeptide or polypeptide fragment, nucleotide sequence, vector or cell of the present invention. The peptides, polypeptides and other compounds and/or compositions of the invention are administered with a pharmaceutically acceptable carrier(s) (excipient) to form the pharmaceutical composition.

Pharmaceutically acceptable carriers and formulations, e.g., for the compounds of the present invention are known to the skilled artisan and are described in detail in the scientific and patent literature, see e.g., the latest edition of Remington's Pharmaceutical Science, Mack Publishing Company, Easton, Pa. (“Remington's”); Banga; Putney (1998) Nat. Biotechnol. 16:153-157; Patton (1998) Biotechniques 16:141-143; Edwards (1997) Science 276: 1868-1871; Ho, U.S. Pat. No. 5,780,431; Webb, U.S. Pat. No. 5,770,700; Goulmy, U.S. Pat. No. 5,770,201.

The compounds or compositions of the present invention can be delivered alone or as pharmaceutical compositions by any means known in the art, e.g., systemically, regionally, or locally; by intraarterial, intrathecal (IT), intravenous (IV), parenteral, intra-pleural cavity, topical, oral, or local administration, as subcutaneous, intra-tracheal (e.g., by aerosol) or transmucosal (e.g., buccal, bladder, vaginal, uterine, rectal, nasal mucosa). Actual methods for delivering compositions will be known or apparent to those skilled in the art and are described in detail in the scientific and patent literature, see e.g., Remington's.

The pharmaceutical compositions can be administered by any protocol and in a variety of unit dosage forms depending upon the method of administration, and the like. Dosages for typical peptide and polypeptide pharmaceutical compositions are well known to those of skill in the art. Such dosages are typically advisorial in nature and are adjusted depending on a variety of factors, such as the particular therapeutic context, patient health and the like. The dosage schedule and amounts effective for this use, i.e., the “dosing regimen,” will depend upon a variety of factors, including the stage of the disease being treated; timing of co-administration of other agents; the general state of the patient's health; the patient's physical status; age; the pharmaceutical formulation, and the like. The dosage regimen also takes into consideration pharmacokinetics, e.g., the peptide pharmaceutical composition's rate of absorption, bioavailability, metabolism, clearance, and the like, see, e.g., Remington.

Dosages can be determined empirically, e.g, by abatement or amelioration of symptoms, or by objective criteria, analysis of blood or histopathology specimens, and the like.

Vectors used for therapeutic administration of modified factor FVII/FVIIa mutant-encoding nucleic acids may be viral or nonviral. Viral vectors are usually introduced into a patient as components of a virus. Illustrative viral vectors into which one can incorporate nucleic acids include, for example, adenovirus-based vectors (Cantwell (1996) Blood 88:4676-4683; Ohashi (1997) Proc. Nat'l. Acad. Sci. USA 94:1287-1292), Epstein-Barr virus-based vectors (Mazda (1997) J. Immunol. Methods 204:143-151), adenovirus-associated virus vectors, Sindbis virus vectors (Strong (1997) Gene Ther. 4: 624-627), herpes simplex virus vectors (Kennedy (1997) Brain 120: 1245-1259) and retroviral vectors (Schubert (1997) Curr. Eye Res. 16:656-662).

Nonviral vectors encoding products useful in gene therapy can be introduced into an animal by means such as lipofection, biolistics, virosomes, liposomes, immunoliposomes, polycation:nucleic acid conjugates, naked DNA injection, artificial virions, agent-enhanced uptake of DNA, ex vivo transduction. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424 and WO 91/16024. Naked DNA genetic vaccines are described in, for example, U.S. Pat. No. 5,589,486.

In accordance with another embodiment of the present invention, a method of treating a mammal having a blood condition is provided, such as hemophilia, or thrombocytopenia, or thrombosis associated with disseminated intravascular coagulation (DIC), or atherosclerosis and cancer. Preferably, a blood condition includes a compromised ability to adequately maintain blood coagulation. A method of treatment as herein provided includes administration of one or more modified form(s) of factor VII/VIIa in vivo. Preferably, a modified form of factor VII/VIIa is in the form of a pharmaceutical composition. According to an alternate embodiment of the present invention, a modified form of factor VII/VIIa is delivered to a mammal in vivo with an expression vector. That is, a modified form of factor VII/VIIa is expressed in vivo by an expression vector appropriately delivered to a recipient in need of the modified form of factor VII/VIIa.

Inactive Forms of Modified Factor VII

As mentioned above, and in view of the prior demonstration of artificially inactivated human rFVIIa (rFVIIai) as an effective inhibitor of thrombosis and death in a baboon model of disseminated intravascular coagulation (DIC) (Taylor F B Jr, Chang A, Peer G, Li A, Ezban M, Hedner U., Blood, 1998, 91: 1609-1615), enzymatically-inactive forms of the high-affinity rFVIIa mutants described herein would provide useful anticoagulants.

Previous studies have established that chloromethylketone (ck) covalent modification of the wild-type(WT) rFVIIa active site renders the rFVIIa enzymatically inactive (rFVIIai) (J. Biol. Chem., 2000, 275:34894). These studies yielded evidence for a linked conformational change of the recombinant factor VII(rFVII) first epidermal growth factor-like(EGF1).

The present inventors have accordingly studied representative examples of the modified rFVIIa enzyme of the present invention, which as described above has increased enzymatic activity and/or affinity for TF, by their TF-binding characteristics, conformational state and plasma clotting activity before and after inactivation using peptide chloromethylketones.

Experiments

Reagents. Purified plasma-derived human factor FVII (pdFVII) and pdFVIIa were from Enzyme Research Labs (South Bend, Ind.). Purified rFVIIa(wt) was purchased from Novo Nordisk Inc. (Bagsvaerd, Denmark) or purified from rFVII(wt)-HEK293 cell line culture medium. Monoclonal antibody (MoAb) 231-7 was expressed and purified as previously described (Leonard B J N, Clarke B J, Sridhara S, Kelley R, Ofosu F A, Blajchman M A, J. Biol. Chem., 2000, 275: 34894-900). Human thromboplastin Thromborel S was a product of Dade Behring (Marburg, Germany). Streptavidin conjugated to alkaline phosphatase was from Jackson Immune Research Laboratories (West Grove, Pa.). Immulon-2HB flat-bottom, 96 well microtiter plates were from VWR Scientific (Mississauga, ON).

Purification of rFVIIai Proteins. Both rFVII(wt) and rFVII(K62E) protein were purified in the zymogen form using a modification of the Q-Sepharose pseudoaffinity chromatography technique as previously described in detail (Williamson V, Pyke A., Sridhara S, Kelley R F, Blajchman, M A, Clarke B J., J. Thromb. Haemost., 2005, 3:1250-56). The purity of the eluted rFVIIs were analyzed by reducing SDS-PAGE and coomassie blue staining for total protein as well as Western blot analysis using biotinylated, polyclonal sheep anti-human rFVII IgG (Williamson et al., J. Thromb. Haemost., 2005, supra). Purified rFVII (250 μg) was quantitatively auto-activated to rFVIIa after equilibration in TBS buffer (20 mM Tris-HCl, pH 8.0, 100 mM NaCl, 0.05% NaN₃) and concentration to 1 ml volume using a centrifugal filtration device (10 kDa molecular weight cut-off, Millipore Corp., Billerica, Mass.). After the addition of 0.25 mM CaCl₂ and 0.25 ml of rehydrated Q-Sepharose resin, the rFVII mixture was gently tumbled in a microfuge tube for 2 hr at room temperature. After brief centrifugation at 15,000×g, the clear supernatant fluid containing rFVIIa was re-equilibrated in 10 ml TBS buffer and re-concentrated by centrifugal filtration to 1 ml. Efficiency of rFVII activation and recovery was determined by reducing SDS-PAGE, prothrombin time (PT) assay and FVII antigen ELISA respectively. Purified rFVIIa was stored in TBS buffer, 10 mM benzamidine at −70° C. Reaction of 50 μg rFVIIa with peptide chloromethylketones was performed in a 1 ml volume of TBS buffer, 2.5 mM CaCl₂, after removal of benzamidine by centrifugal filtration. A 50-fold molar excess of chloromethylketone (10 mm stock in 1 mM HCl) to rFVIIa was added and the reaction incubated at room temperature, for 60 minutes. Two additional aliquots of chloromethylketone were added at one hour intervals. One mg of BSA was then added to the rFVIIa sample as a carrier protein followed by exhaustive dialysis (4×500 ml) versus TBS buffer at room temperature. Efficiency of rFVIIa inactivation and recovery was determined by PT assay and FVII antigen ELISA respectively. Residual coagulant activity of the rFVIIai formed was routinely less than 1%.

Affinity of rFVII for monoclonal antibody 231-7 by competitive ELISA. MoAb 231-7 (100 ng) was adsorbed to each well of a microtiter plate in 100 μl PBS, 25 mM benzamidine, pH 7.4 overnight at 4° C. Following 2× washes with saline the plates were blocked with 200 μl TBS-T+BSA (20 mM Tris-HCl, pH 7.4, 0.15 M NaCl, 5 mM CaCl₂, 0.025% tween-20, 3.5 mg/ml bovine albumin) for 2 hr, at room temperature. Following one wash with TBS-T without BSA, quadruplicate samples in 100 μl of TBS-T+BSA containing both 10 ng/ml biotinylated plasma-derived human VII (probe) and doubling dilutions of competing rFVII (competitor:probe molar ratios varied from 100:1 to 1.5:1) were allowed to bind to the adsorbed MoAb 231-7 for 2 hrs at room temperature. Following four washes with TBS-T w/o BSA containing 1 M NaCl, 100 μl streptavidin-alkaline phosphatase (1:25,000 dilution in TBS-T+BSA) was added for 1 hr, RT. After an additional four washes with TBS-T w/o BSA containing 1M NaCl, the colourimetric reaction was developed with 100 μl of 1 mg/ml p-nitrophenylphosphate in diethanolamine buffer (10% diethanolamine, 0.5 mM MgCl₂, pH 9.8). The reaction was stopped after 60 minutes by the addition of 25 μl of 0.5M EDTA to each well. Absorbance at 405 nm was recorded using an automated microtiter plate reader (EL808, Bio-Tek Instruments, Winooski, Vt.).

Quantitation of FVII Antigen and Total Protein. Total rFVII antigen levels were determined by solid-phase “trap” ELISA employing a polyclonal sheep anti-human rFVII IgG as described (Williamson et al., J. Thromb. Haemost., 2005, supra). Total protein concentrations were determined either by BCA assay (Pierce Scientific Co., Rockford, Ill.) or the Bradford coomassie blue reagent (Sigma-Aldrich, St. Louis, Mo.).

Coagulant Activity of rFVII Mutant Proteins. Coagulant activity of the various FVII samples was measured in quadruplicate by prothrombin time (PT) assay using FVII-depleted human plasma prepared in our laboratory and human thromboplastin reagent essentially as previously described (Clarke B J, Sridhara S., Brit. J. Haematol., 1996, 93:445-450).

Determination of the Relative Affinity of rFVII Mutant Proteins for TF by Competitive ELISA. The binding of biotinylated, plasma-derived FVII to relipidated, full-length recombinant Tissue Factor (rTF) and the quantitation of the relative affinity of rFVII mutant proteins for rTF by inhibition of biotinylated FVII binding (IC50) has been described in detail (Sridhara S, Chaing S, High K A, Blajchman M A, Clarke B J., Amer. J. Hematol., 1996, 53:66-71).

Determination of the Absolute Affinity of rFVII Mutant Protein for rTF by Surface Plasmon Resonance. Binding affinities were determined by surface plasmon resonance using a BIAcore 3000 instrument exactly as previously described (Williamson V, Pyke A., Sridhara S, Kelley R F, Blajchman, M A, Clarke B J., J. Thromb. Haemost., 2005, supra).

Statistical Analysis. Linear regression analysis, t tests, analysis of variance and standard error of the mean were performed using the InStat 3.05 software package (GraphPad Software, San Diego, Calif.) for Windows 98. All experiments were performed three or more times and a representative experiment was selected for statistical analysis.

Results

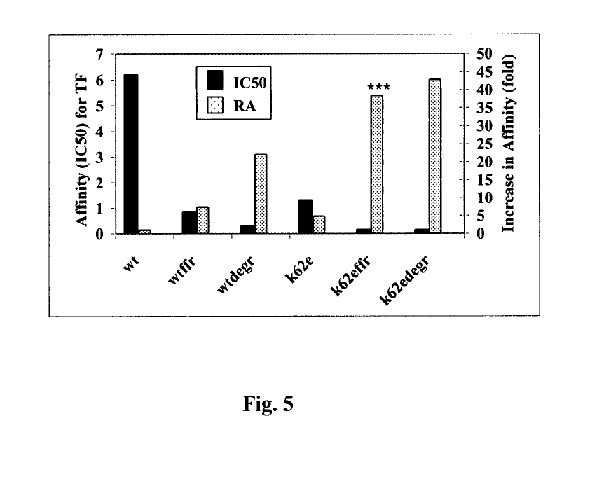

Survey of the effects of various chloromethylketones on the properties of rFVIIa(K62E). Initially we compared the effects of five active-site specific peptide chloromethylketones with differing amino acid residues at the P2 and P3 positions i.e. FFRck, DEGRck, FPRck, PFRck and GGRck on the biological properties of rFVIIa(K62E) and rFVIIa(wt). Indications were that all of the peptide-chloromethylketones increased the affinity of both rFVIIa(K62E) and rFVIIa(wt) for TF however the greatest increases occurred with the substitution of FFRck and DEGRck. FIG. 5 illustrates that the relative affinity of rFVIIa(K62E)-FFR and rFVIIa(K62E)-DEGR for immobilized full-length human TF were increased 38-fold and 43-fold versus rFVIIa(wt). Interestingly, the relative affinity of rFVIIa(K62E) for TF was 4.8-fold greater than rFVIIa(wt), and substitution of both mutant and wild-type rFVIIa with FFR increased their affinity for TF to approximately the same extent. Confirmatory KD measurements (Table 8) demonstrated that rFVIIa(K62E)-FFR bound 4.5-fold more strongly to soluble TF than did rFVIIa(wt)-FFR.

TABLE 8
Absolute affinity of rFVIIa samples for sTF
determined by surface plasmon resonance.

| rFVIIa | Kₒₙ (x10⁵) | K_{off} (x10⁻³) | K_{D} (nM) |
|---|---|---|---|
| rFVIIa (wt) | 1.5 ± 0.3 | 6.1 ± 0.5 | 40.5 ± 7.2 |
| rFVIIa (wt)-FFR | 10.1 ± 3.0 | 1.8 ± 0.4 | 1.8 ± 0.9 |
| rFVIIa (wt)-DEGR | 10.4 ± 3.0 | 1.8 ± 0.1 | 1.4 ± 0.3 |
| rFVIIa (K62E) | 9.0 ± 2.0 | 5.5 ± 0.7 | 6.6 ± 1.7 |
| rFVIIa (K62E)-FFR | 30.0 ± 9.0 | 1.1 ± 0.1 | 0.4 ± 0.1* |
| rFVIIa (K62E)-DEGR | 20.0 ± 3.0 | 1.0 ± 0.1 | 0.5 ± 0.1 |

All data are the mean ± SD, (n = 4 − 6). rFVIIa (wt) was Niastase. The asterisk * indicates a statistically significant difference from rFVIIa (wt)-FFR (P < 0.05) using an unpaired t-test, Welch corrected.

Effect of FFRck and DEGRck inactivation of rFVIIai(K62E) on binding affinity of a conformation-specific monoclonal antibody. MoAb 231-7 has previously been shown to be both specific for the EGF-1 domain of FVII(wt) and sensitive to its conformation (Leonard B J N, Clarke B J, Sridhara S, Kelley R, Ofosu F A, Blajchman M A., J. Biol. Chem., 2000, supra). Competitive ELISA binding data in Table 9 confirmed that both rFVIIa(wt)-FFRck and rFVIIa(wt)-DEGRck are bound with greater affinity by MoAb 231-7 than rFVIIa(wt). MoAb 231-7 bound the mutant rFVIIa(K62E) 4.8-fold more tightly than rFVIIa(wt). Binding of the peptide chloromethylketone derivatized rFVIIa(K62E)-FFR and rFVIIa(K62E)-DEGR to MoAb 231-7 was further enhanced by 1.5-fold and 1.3-fold as compared to unsubstituted rFVIIa(K62E).

TABLE 9
Binding of rFVIIa proteins to the EGF-1 domain,
conformation-sensitive monoclonal antibody 231-7.

| | Relative Affinity | Relative |
|---|---|---|
| | for MoAb231-7 | Increase |
| rFVIIa | (IC50) | in Affinity |
| rFVIIa (wt) | 47.2 ± 8.9 | 1 |
| rFVIIa (wt)-FFR | 33.0 ± 35 | 1.4 |
| rFVIIa (wt)-DEGR | 16.8 ± 3.6** | 2.8 |
| rFVIIa (K62E) | 9.8 ± 2.4*** | 4.8 |
| rFVIIa (K62E)-FFR | 6.5 ± 2.5*** | 7.2 |
| rFVIIa (K62E)-DEGR | 7.8 ± 4.4*** | 6.1 |

The data are the means ± SEM of 5 separate competitive ELISA experiments. The asterisks ** and *** indicate statistically significant differences in affinity from rFVIIa (wt), (P < 0.01 and P < 0.001 respectively) using the Tukey-Kramer multiple comparisons t-test.

Effects of FFRck and DEGRck inactivation of rFVIIa(K62E) and rFVIIa(wt) on coagulant activity of human plasma. In agreement with the substantially increased affinity of rFVIIai(K62E) for TF, rFVIIa(K62E)-FFR and rFVIIa(K62E)-DEGR, are excellent inhibitors of the TF-dependent coagulant activity of human plasma (Table 10). At a 1:1 molar ratio with purified plasma-derived FVII, rFVIIa(K62E)-FFR reduced the coagulant activity to 6% versus 41% for rFVIIa(wt)-FFR. rFVIIa(K62E)-DEGR was also a significantly more potent inhibitor of coagulant activity than rFVIIa(wt)-DEGR but was no more effective than rFVIIa(K62E)-FFR.

TABLE 10
Reduction in coagulant activity by rFVIIai (wt) and rFVIIai (K62E).

| | Coagulant | Residual |
|---|---|---|
| | Activity ^{a} | Coagulant |
| rFVIIai | (U/mg) | Activity (%) |
| rFVIIa (wt)-FFR | 895 ± 105 | 41 |
| rFVIIa (wt)-DEGR | 680 ± 70 | 31 |
| rFVIIa (K62E)-FFR | 125 ± 15*** | 6 |
| rFVIIa (K62E)-DEGR | 290 ± 25*** | 13 |

^{a} Relative to the coagulant activity of FVII in normal pooled plasma (2200 U/mg). All of the rFVIIai were compared at a 1:1 molar ratio with plasma FVII. Results are the mean ± SEM, n = 8.
***Indicates a statistically significant difference from rFVIIa (wt)-FFR, (P < 0.001) using the Tukey-Kramer multiple comparisons t-test.

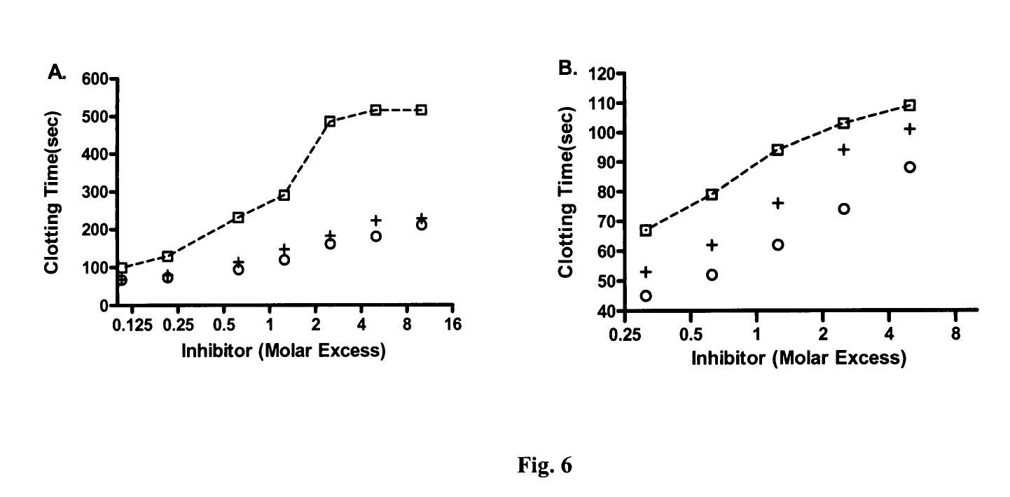

A comparison of the anticoagulant activity of rFVIIai(K62E)-FFR in human and rabbit plasma. FIG. 6 demonstrates that rFVIIa(K62E)-FFR is a superior inhibitor of coagulation versus both rFVIIa(wt)-FFR and pdFVIIa-FFR at molar ratios of inhibitor:plasma FVII as low as 0.25:1 in both human and rabbit plasma. Plasma clotting times are significantly more prolonged by rFVIIa(K62E)-FFR than rFVIIa(wt)-FFR at inhibitor:plasma FVII molar ratios in excess of 1:1. No significant difference in anticoagulant efficacy was observed between rFVIIa(wt)-FFR inhibitors prepared using rFVIIa(wt) purified in our laboratory versus commercial rFVIIa(wt) from Novo Nordisk.

Discussion

The above results demonstrate that modification of the FVII enzyme, for instance by means of a single amino acid substitution K62→E within the amino acid sequence, illicits a significant conformational change in the EGF1 domain of the mutant enzyme versus the rFVIIa(wt). More subtle conformational changes of the EGF1 domain occur after inactivation with peptide chloromethylketone active site inhibitors, such as FFRck, DEGRck, FPRck, PFRck or GGRck, but the inactivated mutant enzymes maintain their higher affinity for moab 231-7 versus rFVIIa(wt). Accordingly, while the peptide chloromethylketone inactivated rFVIIai(wt) molecules bound TF with higher affinity than rFVIIa(wt), the peptide chloromethylketone inactivated mutant rFVIIai molecules, including rFVIIai(K62E)-FFR and rFVIIa(K62E)-DEGR, bound TF significantly more tightly (Table 8, FIG. 5) than their rFVIIai(wt) counterparts. The superior activity of the peptide chloromethylketone inactivated mutant rFVIIai molecules in prolonging the clotting time of both human and rabbit plasma in vitro, as exemplified by rFVIIai(K62E) (Table 10, FIG. 6), confirms the TF binding data. Thus there is a positive correlation between the moab 231-7 binding i.e. the conformation of the EGF1 domain, and the affinity of both rFVIIa(K62E) and rFVIIai(K62E) for TF.

The seminal observation that blockade of the FVIIa-TF axis by rFVIIai abrogated both coagulation and the synthesis of the inflammatory cytokines interleukin-6 and interleukin-8 in experimental DIC in baboons (Taylor F B Jr, Chang A, Peer G, Li A, Ezban M, Hedner U., Blood, 1998, 91:1609-1615) has been confirmed and extended in septic baboons suffering acute lung injury and renal failure (Welty-Wolfe K E, Carraway M S, Miller D L, Ortel T L, Ezban M, Ghio A J, Idell S, Piantadosi C A., Crit. Care Med., 2001, 164:1988-1996). Recent experiments in mice genetically-engineered to produce low levels of either TF (Pawlinski R, Pedersen B, Schabbauer G, Tencati M, Holscher T, Boisvert W, Andrade-Gordon P, Frank R P, Mackman N. Blood, 2004, 103: 1342-1347) or FVII (Xu H, Plopsis V A, Castellini F J., J. Path., 2006, 210:488-496) clearly show that the interaction of FVIIa with TF is integral to the excessive coagulation, acute inflammation and mortality associated with sepsis-induced DIC. In addition, rFVIIai is being actively investigated as an anti-tumour agent. Accordingly, due to their increased affinity for TF and anticoagulation activity, the inactivated mutant rFVIIai molecules described herein provide an improved anticoagulant for treating DIC and/or cancer as compared to the wild type FVIIai. Moreover, the inactivated mutant rFVIIai molecules provide for improved antithrombotics for treatment of thrombosis, atherosclerosis and related disorders.

All references cited herein are incorporated herein by reference to the same extent as if each individual publication, patent application or issued patent was specifically and individually indicated to be incorporated by reference in its entirety for all purposes.

The embodiment(s) of the invention described above is(are) intended to be exemplary only. The scope of the invention is therefore intended to be limited solely by the scope of the appended claims.

### REFERENCES

1. Hagen F S, Gray C L, O'Hara P J, Grant F J, Saari G C, Woodbury R G, Hart C E, Insley M, Kisiel W, Kurachi K, Davie E W. Characterization of a cDNA coding for human factor VII. Proc Natl Acad Sc. USA 1986; 83: 2412-2416.
2. Broze G J Jr. Tissue factor pathway inhibitor and the revised theory of coagulation. Annu Rev Med 1995; 46: 103-112.
3. Hockin M F, Jones K C, Everse S J, Mann K G. A model for the stoichiometric regulation of blood coagulation. J Biol Chem 2002; 277: 18322-18333.
4. Versteeg H H, Peppelenbosch M P, Spek C A. The pleiotrophic effects of tissue factor: a possible role for factor VIIa-induced intracellular signalling. Thromb Haemost 2001; 86: 1353-1359.
5. Ruf W, Dorfleutner A, Riewald M. Specificity of coagulation factor signalling. J Thromb Haemost 2003; 1: 1495-1503.
6. Chen J, Bierhaus A, Schiekofer S, Andrassy M, Chen B, Stern D M, Nawroth P P. Tissue factor—a receptor involved in the control of cellular properties, including angiogenesis. Thromb Haemost 2001; 86: 334-345.
7. Konigsberg W, Kirchhofer D, Riederer M A, Nemerson Y. The TF:VIIa complex: clinical significance, structure-function relationships and its role in signalling and metastasis. Thromb Haemost 2001; 86:757-771.
8. Taylor F B Jr, Chang A, Peer G, Li A, Ezban M, Hedner U. Active site inhibited factor VIIa(DEGR VIIa) attenuates the coagulant and interleukin-6 and -8 but not tumor necrosis factor responses of the baboon to LD₁₀₀ Escherichia coli. Blood 1998; 91: 1609-1615.
9. Kelley R F, Yang J, Eigenbrot C, Moran P, Peek M, Lipari M, Kirchhofer D. Similar molecular interactions of factor VII and factor VIIa with the tissue factor region that allosterically regulates enzyme activity. Biochem 2004; 43: 1223-1229.

10. Presta L, Sims P, Meng Y G, Moran P, Bullens S, Bunting S, Schoenfeld J, Lowe D, Lai J, Rancatore P, Iverson M, Lim A, Chisholm V, Kelley R F, Riederer M, Kirchhofer D. Generation of a humanized, high-affinity anti-tissue factor antibody for use as a novel therapeutic. Thromb Haemost 2001; 85: 379-389.
11. Yang J, Lee G F, Riederer M A, Kelley R F. Enhancing the anticoagulant potency of soluble tissue factor mutants by increasing their affinity to factor VIIa. Thromb Haemost 2002; 87: 450-458.
12. Dennis M S, Eigenbrot C, Skeleton N J, Ultsch M H, Santell L, Dwyer M A, O'Connell M P, Lazarus R A. Peptide exosite inhibitors of factor VIIa as anticoagulants. Nature 2000; 404: 465-470.
13. Maun H R, Eigenbrot C, Lazarus R A. Engineering exosite peptides for complete inhibition of factor VIIa using a protease switch with substrate phage. J Biol Chem 2003; 278: 21823-21830.
14. Chaing S, Clarke B J, Sridhara S, Chu K, Friedman P, Van Dusen W, Roberts H R, Blajchman M, Monroe D M, High K A. Severe factor VII deficiency caused by mutations abolishing the cleavage site for activation and altering binding to tissue factor. Blood 1994; 83: 3524-3535.
15. Leonard B L, Chen Q, Blajchman M A, Ofosu F A, Sridhara S, Yang D, Clarke B J. Factor VII deficiency caused by a structural variant N57D of the first epidermal growth factor domain. Blood 1998; 91: 142-148.
16. McVey J H, Boswell E, Mumford A D, Kemball-Cook G, Tuddenham E G D. Factor VII deficiency and the factor VII mutation database. Human Mutation 2001; 17: 3-17.
17. Dickinson C D, Kelly C R, R W. Identification of surface residues mediating tissue factor binding and catalytic function of the serine protease factor VIIa. Proc Natl Acad Sci USA 1996; 93: 14379-14384.
18. Janson T L, Stormorken H, Prydz H. Species specificity of tissue thromboplastin. Haemostasis 1984; 14: 440-444.
19. Banner D W, D'Arcy A, Chene C, Winkler F K, Guha A, Konigsberg W H, Nemerson Y, Kirchhofer D. The crystal structure of the complex of factor VIIa with soluble tissue factor. Nature 1996; 380: 41-46.
20. Ruiz S, Sridhara S, Blajchman A, Clarke B J. Expression and purification of recombinant rabbit factor II. Thromb Res 2000; 98: 203-211.
21. Williamson V, Pyke A, Sridhara, S, Blajchman M A, Clarke B J. Interspecies exchange mutagenesis of the first epidermal growth factor-like domain of human factor VII. J Thromb Haemost 2003; Suppl 1: abstract PO 559.
22. Clarke B J, Sridhara S. Incomplete gamma carboxylation of human coagulation factor VII: differential effects on tissue factor binding and enzymatic activity. Brit J Haematol 1996; 93: 445-450.
23. Yan S C B, Razzano P, Chao B, Walls J, Berg D, McClure D, Grinnell B. Characterization and novel purification of recombinant human protein C from three mammalian cell lines. Bio/technology 1990; 8: 655-661.
24. Sridhara S, Chaing S, High K A, Blajchman M A, Clarke B J. Activation of a recombinant human factor VII structural variant alters its affinity of binding to tissue factor. Amer J Hematol 1996; 53: 66-71.
25. Kelley R F, Costas K E, O'Connell M P, Lazarus R A. Analysis of the factor VIIa binding site on human tissue factor: effects of tissue factor mutations on the kinetics and thermodynamics of binding. Biochem 1995; 34: 10383-10392.
26. Clarke B J, Ofosu F A, Sridhara S, Bona R D, Rickles F R, Blajchman M A. The first epidermal growth factor domain of human coagulation factor VII is essential for binding to tissue factor. Febs Letts 1992; 298: 206-210.
27. Chang J Y, Stafford D W, Straight D L. The roles of factor VII's structural domains in tissue factor binding. Biochem 1995; 34: 12227-12232.
28. Clarke B J, Sridhara S, Blajchman M A, Hatton M W C. Inhibition of the rabbit extrinsic pathway of coagulation: A comparison of rabbit and human FVII structural analogues. Can J Cardiol 2003; 18: (Suppl B): 202B, abstract 331.
29. Kelley R F, Refino C J, O'Connell M P, Modi N, Sehl P, Lowe D, Pater C, Bunting S. A soluble tissue factor mutant is a selective anticoagulant and antithrombotic agent. Blood 1997; 89: 3219-3227.
30. O'Brien D P, Kemball-Cook G, Hutchinson A M, Martin D M A, Johnson D J D, Byfield P G H, Takamiya O, Tuddenham E G D, McVey J H. Surface plasmon resonance studies of the interaction between factor VII and tissue factor. Biochem 1994; 33: 14162-14169.
31. Davidson C J, Tuddenham E G, McVey J H. 450 million years of hemostasis. J Thromb Haemost 2003; 1: 1487-1494.
32. Seetharam S, Murphy K, Atkins C, Feuerstein G. Cloning and expression of rat coagulation factor VII. Thromb Res 2003; 109: 225-231.
33. Leonard B J N, Clarke B J, Sridhara S, Kelley R, Ofosu F A, Blajchman M A. Activation and active site occupation alter conformation in the region of the first epidermal growth factor-like domain of human factor VII. J Biol Chem 2000; 275: 34894-34900.
34. Persson E, Olsen O H, Ostergaard A, Nielsen L S. Ca²⁺ binding to the first epidermal growth factor-like domain of factor VIIa increases amidolytic activity and tissue factor affinity. J Biol Chem 1997; 272: 19919-19924.
35. Jin J, Perera L, Stafford D, Pedersen L. Four loops of the catalytic domain of factor VIIa mediate the effect of the first EGF-like domain substitution on factor VIIa catalytic activity. J MolBiol 2001; 307:1503-1517.
36. Dickinson C D, Shobe J, Ruf W. Influence of cofactor binding and active site occupancy on the conformation of the macromolecular substrate exosite of factor VIIa. Mol Biol 1998; 277: 959-971.
37. Persson E. Variants of recombinant factor VIIa with increased intrinsic activity. Semin Hematol 2004; 41(Suppl 1): 89-92.
38. Persson E, Kjalke M, Olsen O H. Rational design of coagulation factor VIIa variants with substantially increased intrinsic activity. Proc Natl Acad Sci USA 2001; 98: 13583-13588.
39. Hedner U. Recombinant factor VIIa (Novoseven) as a hemostatic agent. Semin Hematol 2001; 38(Suppl 12): 43-47.
40. Butenas S, Brummel B, Mann K G. How factor VIIa works in hemophilia. J Thromb Haemost 2003; 1: 1158-1160.
41. Sorensen B B, Persson E, Freskgard P O, Kjalke M, Ezban M, Williams T, Rao L V M. Incorporation of an active site inhibitor in factor VIIa alters the affinity for TF. J Biol Chem 1997; 272: 11863-11868.
42. Sorensen B B, Rao L V, Tornehave D, Gammeltoft S, Petersen L C. Antiapoptotic effect of coagulation factor VIIa. Blood 2003; 102: 1708-1715.
43. Jiang X, Bailly M A, Panetti T S, Cappello M, Konigsberg W H, Bromberg M E. Formation of tissue factor-factor Xa complex promotes cellular signalling and migration of human breast cancer cells. J Thomb Haemost 2003; 2: 93-101.
44. Versteeg H H, Spek C A, Richel D J, Peppelenbosch M P. Coagulation factors VIIa and Xa inhibit apoptosis and anoikis. Oncogene 2004; 23: 410-417.
45. Butenas S, Brummel B, Mann K. How factor VIIa works in hemophilia. J Thromb Haemost 2003; 1:1158-1160. THIS REF REPLACES VANT VEER.
46. Roberge M, Peek M, Kirchhofer D, Dennis M S, Lazarus R A. Fusion of two distinct peptide exosite inhibitors of factor VIIa. Biochem J 2002; 263:387-393.

I/We claim:

1. An inactivated factor VII/VIIa mutant or functional fragment thereof, comprising one or more mutation(s) in the epidermal growth factor-like (EGF-1) domain.

2. The inactivated FVII/FVIIa mutant according to claim 1, wherein said mutation(s) is/are at one, more than one, or all amino acid residues at positions 53, 62, 74, 75, 83, or any combination thereof, wherein said mutation(s) may be to any amino acid residue that confers enhanced biological activity of said FVII/FVIIa.

3. The inactivated FVII/FVIIa mutant according to claim 1, wherein said mutant FVII/FVIIa comprises one, more than one or all mutations selected from (S53N), (K62E), (K62D), (K62N), (K62Q), (K62T), (P74A), (A75D) and (T83K).

4. The inactivated FVII/FVIIa mutant according to claim 1, wherein said mutant FVII/FVIIa comprises mutation (K62E) or mutation (K62T).

5. The inactivated FVII/FVIIa mutant according to claim 1, wherein said mutant FVII/FVIIa comprises an EGF-1 domain encoded by a nucleic acid sequence selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9 and SEQ ID NO: 10, or any degenerate variant thereof.

6. The inactivated FVII/FVIIa mutant according to claim 1, wherein said mutant FVII/FVIIa comprises an EGF-1 domain comprising a polypeptide sequence selected from SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19 and SEQ ID NO: 20.

7. The inactivated FVII/FVIIa mutant according to claim 1, inactivated by treatment with an irreversible active site inhibitor.

8. The inactivated FVII/FVIIa mutant according to claim 7, wherein the irreversible active site inhibitor is a peptide chloromethylketone.

9. The inactivated FVII/FVIIa mutant according to claim 8, wherein the peptide chloromethylketone is selected from FFR-ck, DEGR-ck, FPR-ck, PFR-ck and GGR-ck.

10. The inactivated FVII/FVIIa mutant according to claim 8, wherein the peptide chloromethylketone is FFR-ck.

11. The inactivated FVII/FVIIa mutant according to claim 8, wherein the peptide chloromethylketone is DEGR-ck.

12. The inactivated FVII/FVIIa mutant according to claim 4, inactivated by treatment with an irreversible active site inhibitor.

13. The inactivated FVII/FVIIa mutant according to claim 12, wherein the irreversible active site inhibitor is a peptide chloromethylketone.

14. The inactivated FVII/FVIIa mutant according to claim 13, wherein the peptide chloromethylketone is selected from FFR-ck, DEGR-ck, FPR-ck, PFR-ck and GGR-ck.

15. The inactivated FVII/FVIIa mutant according to claim 13, wherein the peptide chloromethylketone is FFR-ck.

16. The inactivated FVII/FVIIa mutant according to claim 13, wherein the peptide chloromethylketone is DEGR-ck.

17. A pharmaceutical composition comprising an inactivated factor VII/VIIa mutant or functional fragment thereof, said mutant comprising one or more mutation(s) in the epidermal growth factor-like (EGF-1) domain, and a pharmaceutically acceptable carrier.

18. The pharmaceutical composition according to claim 17, wherein said mutation(s) is/are at one, more than one, or all amino acid residues at positions 53, 62, 74, 75, 83, or any combination thereof, wherein said mutation(s) may be to any amino acid residue that confers enhanced biological activity of said FVII/FVIIa.

19. The pharmaceutical composition according to claim 17, wherein said mutant FVII/FVIIa comprises one, more than one or all mutations selected from (S53N), (K62E), (K62D), (K62N), (K62Q), (K62T), (P74A), (A75D) and (T83K).

20. The pharmaceutical composition according to claim 17, wherein said mutant FVII/FVIIa comprises mutation(K62E) or mutation (K62T).

21. The pharmaceutical composition according to claim 17, wherein said mutant FVII/FVIIa comprises an EGF-1 domain encoded by a nucleic acid sequence selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9 and SEQ ID NO: 10, or any degenerate variant thereof.

22. The pharmaceutical composition according to claim 17, wherein said mutant FVII/FVIIa comprises an EGF-1 domain comprising a polypeptide sequence selected from SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19 and SEQ ID NO: 20.

23. The pharmaceutical composition according to claim 17, wherein the FVII/FVIIa mutant is inactivated by treatment with an irreversible active site inhibitor.

24. The pharmaceutical composition according to claim 23, wherein the irreversible active site inhibitor is a peptide chloromethylketone.

25. The pharmaceutical composition according to claim 23, wherein the peptide chloromethylketone is selected from FFR-ck, DEGR-ck, FPR-ck, PFR-ck and GGR-ck.

26. The pharmaceutical composition according to claim 23, wherein the peptide chloromethylketone is FFR-ck.

27. The pharmaceutical composition according to claim 23, wherein the peptide chloromethylketone is DEGR-ck.

28. The pharmaceutical composition according to claim 20, wherein the FVII/FVIIa mutant is inactivated by treatment with an irreversible active site inhibitor.

29. The pharmaceutical composition according to claim 28, wherein the irreversible active site inhibitor is a peptide chloromethylketone.

30. The pharmaceutical composition according to claim 29, wherein the peptide chloromethylketone is selected from FFR-ck, DEGR-ck, FPR-ck, PFR-ck and GGR-ck.

31. The pharmaceutical composition according to claim 29, wherein the peptide chloromethylketone is FFR-ck.

32. The pharmaceutical composition according to claim 29, wherein the peptide chloromethylketone is DEGR-ck.

33. A method for treating or preventing thrombosis, stroke, atherosclerosis, disseminated intravascular coagulation (DIC) or cancer, comprising administering a pharmaceutically effective amount of a composition according to any one of claims 17 to 32.
